(19) United States
(12) Patent Application Publication
(10) Pub. No.: US 2019/0134276 A1
(43) Pub. Date: May 9, 2019

(54) THREE DIMENSIONAL POROUS CARTILAGE TEMPLATE

(71) Applicant: Drexel University, Philadelphia, PA (US)

(72) Inventors: Kara Lorraine Spiller, Glenside, PA (US); Nathan Tessema Ersumo, Philadelphia, PA (US)

(73) Assignee: Drexel University, Philadelphia, PA (US)

(21) Appl. No.: 16/310,576

(22) PCT Filed: Jun. 22, 2017
(86) PCT No.: PCT/US17/38718

§ 371 Date: Dec. 17, 2018

Related U.S. Application Data
(60) Provisional application No. 62/353,799, filed on Jun. 23, 2016.

(51) Int. Cl. A61L 27/56; C09D 11/04; A61L 27/54; A61L 27/52; A61L 27/38; A61L 27/22; A61F 2/46; A61F 2/44; C09D 11/101

(52) U.S. Cl. CPC A61L 27/56; C09D 11/04; A61L 27/54; A61L 27/52; A61L 27/3834; B33Y 80/00; A61F 2/46; A61F 2/44; C09D 11/101; A61L 2430/02; A61L 2300/64; A61L 27/222

(57) ABSTRACT

This application relates to biologically compatible porous cartilage templates for in vitro and in vivo generation of bone with enhanced structural characteristics. Provided herein are compositions having an internal structure desirable for the generation and regeneration of bone, along with methods of preparation and use.

### CROSS REFERENCE TO RELATED APPLICATIONS

The present application claims priority to U.S. Provisional Patent Application No. 62/353,799, filed Jun. 23, 2016, which is incorporated herein by reference in its entirety.

### STATEMENT REGARDING FEDERALLY SPONSORED RESEARCH OR DEVELOPMENT

This invention was made with government support under Grant No. R01 HL130037 awarded by the National Institutes of Health. The U.S. Government has certain rights in the invention.

### BACKGROUND OF THE INVENTION

Although bone has an exceptional capacity for regeneration, repairing severe bone defects and fractures remains a critical challenge. Every year, over 600,000 cases linked to cancer or traumatic injury require the use of bone grafting, generating an annual cost of $2.5 billion. These pre-formed grafts, which are either autogeneic or allogeneic, are associated with a number of complications including donor site morbidity for autografts and immune rejection for allografts.

Metal implants, including those coupled with osseointegrative methods including surface functionalization/coating and therapeutic release, constitute one area of investigation. Another active area of investigation has been the development of bioresorbable polymer scaffolds as potential grafts. Myriad approaches have been considered (ex: ceramic vs hydrogel materials, cell-laden vs cell-coated polymers, release vs immobilization of growth factors).

Concomitant with the prevalence of bone defects, current population trends have also led to an increased incidence of other bone-related diseases. One salient example is osteoporosis, a disease characterized by decreased bone mineral density resulting in increased risk of fracture, which affects 2-8% of males and 9-38% of females in developed countries. Other conditions include osteogenesis imperfecta, a congenital disorder characterized by brittle bones, and Paget's disease of bone, a chronic disorder caused by disorganized bone remodeling. Bone tissue is also susceptible to malignant growths and metastases from surrounding organs. Research into the pathologies behind these conditions, which are not yet fully understood, as well as testing for potential therapeutic drugs remain largely centered around in vivo studies, namely animal models and clinical trials.

### SUMMARY OF THE INVENTION

In one aspect the invention provides an engineered porous cartilage template having a bone-mimicking internal structure.

In various embodiments, the porous cartilage template comprises a network of interconnected rod elements and plate elements, wherein the Structural Model Index of said template ranges between 0 and 3, exclusive.

In various embodiments at least 90% of the plate elements have a volume range between 4×10⁶ μm³ and 30×10⁶ μm³, inclusive.

In various embodiments at least 90% of the rod elements have a volume range between 2×10⁶ μm³ and 15×10⁶ μm³, inclusive.

In various embodiments at least 90% of the plate elements have a thickness between 50 μm and 200 μm, inclusive.

In various embodiments at least 90% of the rod elements have a thickness between 50 and 110 μm, inclusive.

In various embodiments at least 90% of the rod elements have a geometric tortuosity range between 1 and 2.5, inclusive.

In various embodiments the separation range between any two elements is between 0.3 and 1.7 mm, inclusive.

In various embodiments the numeric density range for all elements is between 0.5 and 3 mm⁻¹, inclusive.

In various embodiments the numeric density range for the plate elements is between 1.1 and 2.5 mm⁻¹, inclusive.

In various embodiments the numeric density range for the rod elements is between 1.6 and 2.6 mm⁻¹, inclusive.

In various embodiments the rod-rod connectivity density is between 0.5 and 8 mm³, inclusive.

In various embodiments the plate-plate connectivity density is between 2 and 35 mm³, inclusive.

In various embodiments the rod-plate connectivity density is between 3 and 35 mm³, inclusive.

In various embodiments the porous cartilage template has a porosity is between 30% and 90%, inclusive.

In various embodiments the porous cartilage template has a surface-to-volume ratio is between 5 and 25 mm²/mm³, inclusive.

In various embodiments the template comprises a hydrogel matrix.

In various embodiments said hydrogel matrix is gelatin.

In various embodiments, the invention provides a composition comprising the porous cartilage template and mesenchymal stem cells (MSCs).

In various embodiments, said mesenchymal stem cells are encapsulated within said template.

In various embodiments, said mesenchymal stem cells are coated on said template.

In various embodiments, the invention provides a composition comprising the porous cartilage template and chondrocytes.

In various embodiments, said chondrocytes are encapsulated within said template.

In various embodiments, said chondrocytes are coated on said template.

In various embodiments, the porous cartilage template further comprises a bioactive agent.

In various embodiments, the bioactive agent is an RGDS peptide or cartilage oligomeric matrix protein (COMP).

In another aspect, the invention provides a method of promoting the repair of a bone defect in a patient, the method comprising preparing a porous cartilage template having a bone-mimicking internal structure, embedding a plurality of cells into the porous cartilage template, and implanting the porous cartilage template into the bone defect in the patient, thereby promoting the repair of the bone defect.

In various embodiments, method further comprises a step of stabilizing the bone defect.

In various embodiments the step of stabilizing the bone defect comprises emergency surgery to immobilize the bone defect by the insertion of one or more selected from the group consisting of: compression plates, rods, nails, Kirschner wires, and casts.

In various embodiments, the porous cartilage template is prepared by 3D-printing.

In various embodiments, 3D-printing is based on imaging data acquired from a bone defect in the patient.

In various embodiments, the imaging data is acquired by computed tomography (CT) scan or magnetic resonance imaging.

In various embodiments, the plurality of cells comprises mesenchymal stem cells.

In various embodiments, the mesenchymal stem cells are harvested from the patient.

In various embodiments, the plurality of cells comprises chondrocytes.

In various embodiments, the 3D-printing and embedding steps are performed simultaneously.

In various embodiments, the plurality of cells is contained in a hydrogel that is 3D-printed to form at least a portion of the porous cartilage template.

In various embodiments, the method further comprises culturing the plurality of cells to produce mature cartilage.

In various embodiments, the plurality of cells are mesenchymal stem cells and further comprising differentiating the mesenchymal stem cells into chondrocytes.

In various embodiments, the porous cartilage template is secured in the bone defect by press fitting.

In another aspect, the invention provides a method of preparing a porous cartilage template for bone repair, the method comprising: 3D-printing a porous network based on bone imaging data, the porous network comprising: a support component; a sacrificial component; and a plurality of pores; casting a cell-carrier component comprising a plurality of cells into the plurality of pores, evacuating the sacrificial component to form a network of passages among the support component and cell-carrier component; and culturing the plurality of cells of cells to form mature cartilage; thereby forming the porous cartilage template.

In various embodiments, support component comprises polycaprolactone.

In various embodiments, the sacrificial component has a melting point of about 65° C.

In various embodiments, the sacrificial component is polyethylene glycol 20,000.

In various embodiments, the plurality of cells comprises mesenchymal stem cells.

In various embodiments, the step of culturing comprises differentiating the mesenchymal stem cells into chondrocytes.

In various embodiments, the cell carrier component is a hydrogel.

In various embodiments, the hydrogel comprises gellan gum and gelatin.

In various embodiments, the hydrogel further comprises a bioactive agent.

In various embodiments, the bioactive agent is an RGDS peptide or cartilage oligomeric matrix protein (COMP).

In various embodiments, the method further comprises a step of crosslinking the cell-carrier component.

In various embodiments, the step of crosslinking the cell-carrier component comprises exposing the cell-carrier component to a chemical crosslinker.

In various embodiments, the cell-carrier component comprises a solution containing 0.75% w/v gellan gum and 0.25% w/v gelatin, and wherein the chemical crosslinker is calcium chloride.

In various embodiments, the sacrificial component is evacuated by dissolution in aqueous solution.

### BRIEF DESCRIPTION OF THE DRAWINGS

FIG. 1 represents a computer aided design (CAD) structure of bone.

FIG. 2A is a graph of a representative portion of stress/strain data from unconfined compression.

FIG. 2B is a bar chart of Young's moduli of printed and molded GelMA cylinders (15% GelMA, 0.25% LAP).

FIG. 2C depicts the Young's moduli of printed and molded GelMA cylinders (15% GelMA, 0.25% LAP). Note that elastic moduli in FIG. 2B were measured in uniaxial compression with a strain rate of 10%/min, while elastic moduli in FIG. 2C were measured using a strain rate of 16.5%/min. Together, FIGS. 2A-C show that elastic deformation behavior is modified by biomaterial composition but not by printing itself.

FIGS. 3A-3E show that printing affects rate and extent of time-dependent mechanical behavior. Printed and molded GelMA cylinders (15% GelMA, 0.25% LAP) were subjected to creep testing in hydrated and unconfined compression. FIG. 3A is a graph with representative strain vs. time data shown for creep+recovery testing of printed cylinders. FIG. 3B is a bar chart with creep extent data, obtained from exponential regression of creep portion (** p<0.01). FIG. 3C is a bar chart with creep rate data, obtained from exponential regression of creep portion (****p<0.0001). FIG. 3D is a bar chart with recovery extent data, obtained from exponential regression of recovery portion. FIG. 3E is a bar chart with recovery rate data, obtained from exponential regression of recovery portion (**p<0.01).

FIGS. 4A-4D show that printed and molded hydrogels exhibit differential microstructures as well as swelling behavior. Optical micrographs for molded (FIG. 4A) and printed (FIG. 4B) GelMA samples (15% GelMA, 0.25% LAP). Scale bars 500 μm. FIG. 4C is a plot of swelling percentage data (*p<0.05, **p<0.01) obtained from weighing printed and molded GelMA cylinders (15% GelMA, 0.25% LAP) over time in immersion in PBS. FIG. 4D depicts Swelling percentage data (*p<0.05, **p<0.01) obtained from weighing printed and molded GelMA cylinders (15% GelMA, 0.25% LAP) over time in immersion in PBS.

FIG. 5 depicts a proposed approach to critical bone defect repair related to certain embodiments of the invention. (a) A patient suffering from a long bone defect first undergoes an emergency surgery to immobilize the defect area (using compression plates, rods, nails, casts). 3D imaging outlining defect boundaries (ex: CT scanning) is performed. In addition, the patient's mesenchymal cells are harvested from adipose tissue through liposuction and differentiated into chondrocytes. (b) The boundary conditions and obtained chondrocytes are employed to construct a customized cartilage template by printing a hybrid scaffold, consisting of a stiff support structure and a cell-laden hydrogel network, and subsequently culturing the scaffold for tissue maturation. (c) The generated graft is implanted to the defect area and immobilized using press fitting, made possible by the stiff network within the scaffold, and compression plates. Following successful integration and ossification of the fabricated graft, compression plates are removed, leaving a fully healed long bone devoid of foreign material.

FIG. 6 depicts experimental design for various examples discussed below. (a) Various formulations of gelatin methacrylate (GelMA) hydrogel photocrosslinked with lithium phenyl-2,4,6-trimethylbenzoylphosphinate (LAP) were extruded into different structures (b) at varying travel feed rates, nozzle diameters and extrusion pressures (c). (d) Hydrogels with the same dimensions were prepared, and (e) hydrated unconfined compression testing, a swelling study, and optical microscopy were used to evaluate construct properties for comparison against molded counterparts prepared with the exact same dimensions. Scale bars: 5 mm.

FIG. 7 depicts optimal extruding pressure is dependent on biomaterial composition. (a) Sequential lines, as shown by CADmodel, were extruded at various concentrations (10%, 15% and 20% w/v GelMA) and pressures (0-140 psi) with a 27 G nozzle. LAP concentration was 0.5% w/v and travel feed rate was 8 mm s−1. Micrographs shown are representative of line extrusions at 60 psi (b), 80 psi (c) and 100 psi (d) for 10% GelMA/0.25% LAP. Scale bars: 1000 μm.

FIG. 8A depicts representative images shown for line extrusions at 4 mm s−1 (left), 8 mm s−1 (center) and 12 mm s−1 (right) for 10% GelMA/0.25% LAP. Scale bars: 1000 μm.

FIG. 8B depicts representative images shown for line extrusions with a 22 G nozzle at the optimal pressure of 40 psi (left) and with an 18 G nozzle at the optimal pressure of 10 psi (right) for 10% GelMA/0.25% LAP and a travel feed rate of 8 mm s−1. Scale bars: 1000 μm.

FIG. 8C depicts line thickness data as a function of GelMA concentration (10% or 20%) and travel feed rate, quantified by micrograph analysis (****p≤0.0001, two way ANOVA and Tukey post hoc analysis).

FIG. 8D depicts line thickness data as a function of nozzle gauge quantified by micrograph analysis (**p≤0.01, ****p≤0.0001, one way ANOVA and Tukey post hoc analysis).

FIG. 9A depicts cell viability was not affected by 3D-printing process. (a)-(c) 3D-printed hydrogel lines; (d)-(f) molded hydrogels; (g)-(i) cell-only controls.

FIG. 9B depicts quantitative analysis of cell viability. Scale bars are 100 μm.

FIG. 10A depicts how, in various embodiments, a porous hybrid construct is printed by interweaving crosshatch networks of PCL (gray) and PEG (orange) in a repeating PCL strut-pore channel-PCL strut-PEG strut pattern and immersed in a non-crosslinked, composite GG/gelatin solution (blue) to fill the primary porous network. The construct is subsequently immersed in culture media containing Ca²⁺ in order to crosslink the solution into a hydrogel and dissolve away the PEG network, creating a secondary porous network. Characterization of final, sectioned constructs included geometry analysis by photography, porosity analysis using micro-CT scans, mechanical testing and a swelling study.

FIG. 10B depicts established nomenclature of construct experimental groups as classified by hydrogel channel thickness and pore channel thickness. Percentages indicated correspond to construct porosities.

FIG. 10C depicts computer models of all four experimental groups. Generated constructs consisted of 10 layers, each with a height of 0.5 mm, and had bulk dimensions of 5 mm×5 mm×5 mm. PCL struts had widths of 1 mm. Both the widths of the primary pore channels to be filled with hydrogel material and the PEG struts to be dissolved away forming secondary pore channels were varied to values of 0.5 mm and 1 mm.

FIG. 11 depicts a photographic evaluation of constructs from all experimental groups immediately after printing from top (a-d) and isometric views (e-h) as well as after sectioning into individual samples (i-l). All scale bars: 0.5 mm.

FIG. 12A depicts 3D images rendered from micro-CT scanning of 1P/1HG samples at different stages of preparation confirm complete hydrogel suffusion into the primary porous network as well as the dissolution of the sacrificial PEG network, leading to the formation of a secondary porous network. (a) Scan of 1P/1HG construct immediately after extrusion. (b) Scan of 1P/1HG construct immersed in culture media after extrusion. (c) Scan of 1P/1HG construct immersed in hydrogel solution after extrusion. (d) Scan of 1P/1HG construct immersed in hydrogel solution and subsequently in culture media.

FIG. 12B is a graph that depicts porosity values of the 1P/1HG construct at each stage of preparation as expected from designs and as measured from generated micro-CT scans.

FIG. 13A depicts representative stress vs time curve obtained from stress relaxation testing protocol.

FIG. 13B depicts Young's moduli of constructs from all experimental groups.

FIG. 13C depicts total stress relaxation of constructs from all experimental groups.

FIG. 13D depicts τ value of stress relaxation of constructs from all experimental groups.

FIG. 13E depicts relaxation percentage of constructs from all experimental groups.

FIG. 13F depicts relaxation rate of constructs from all experimental groups.

FIG. 14 depicts hydrogels in 0P/1HG constructs and weight-matched plain hydrogel control exhibit different swelling behavior. Swelling percentage data (*p≤0.05, **p≤0.01, ***p≤0.001) obtained from weighing hydrogels immersed in culture medium over time.

FIG. 15 depicts the binding of fluorescent streptavidin to biotinylated gelatin hydrogel (left) but not to non-biotinylated gelatin hydrogel (right).

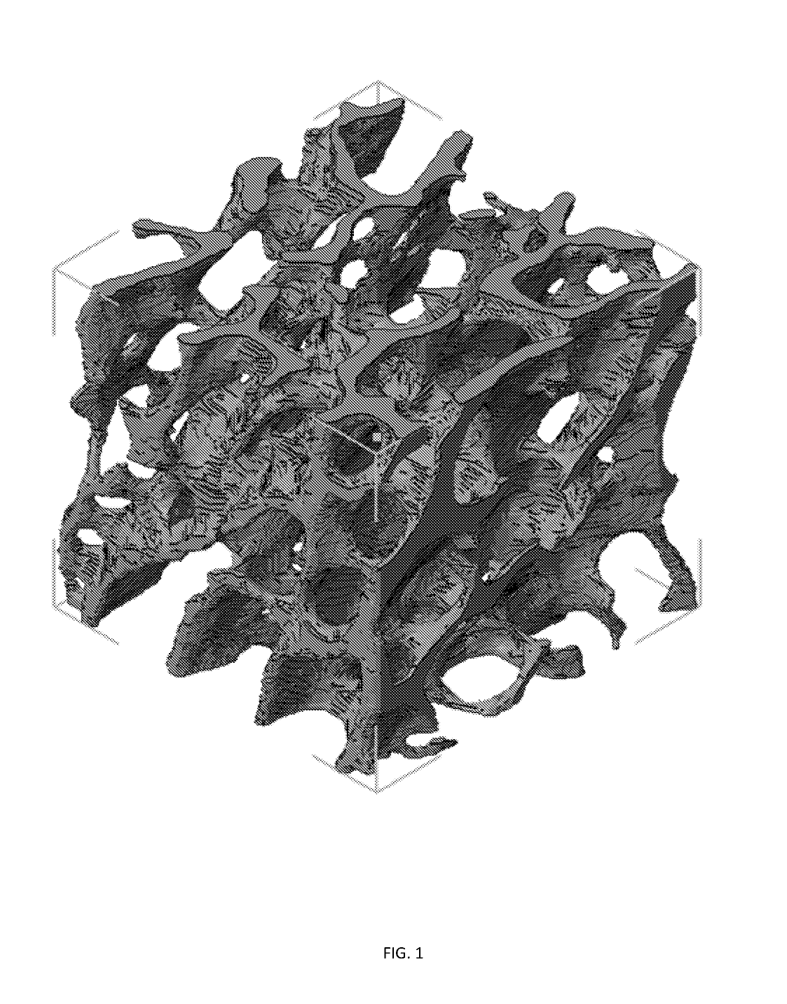

### DETAILED DESCRIPTION OF THE INVENTION

Existing scaffolds or implants for bone generation or regeneration are flawed. These materials are non-degradable and thus preclude the possibility for full repair through resorption and regeneration. Limitations also exist in achieving the balance between structure, mechanical behavior and function needed to ensure both load bearing requirements upon implantation and susceptibility to resorption for later bone regeneration. Thus, there is a need for better bone-regenerating grafts.

Current in vitro models of bone, which include three-dimensional (3D) cultures using microfluidics and ceramic scaffolds, lack the physiological relevance to constitute a viable platform for research. However, the disconnect between the multitude of potential avenues of investigation and the resource/safety considerations of in vivo studies warrants the need for a versatile in vitro bone model capable of recapitulating native tissue as well as diseased states. Such a model could, for instance, establish a high throughput drug screening platform which may be used as a precursor to in vivo studies.

### Porous Cartilage Template

This application provides 3D porous cartilage templates, which overcome the drawbacks of prior constructs and methods. Development and repair of long bones occur through endochondral ossification, in which mesenchymal stem cells (MSCs) differentiate into chondrocytes and form a cartilage template with pores and canals to guide invading capillaries. Infiltrating blood vessels bring immune cells that degrade the cartilage model, which is then replaced by trabecular bone.

Bone microstructure has been shown to affect stress distribution and the effects of regional mechanical stresses on endochondral ossification have previously been demonstrated extensively. Taken together, these findings underline the pivotal role of structure for cartilage templates in their outcome vis-à-vis ossification.

The inventions described herein address the lack of control over structure of previous technologies by developing a porous bone-like cartilage template in order to recapitulate stress distributions observed in native tissue during endochondral ossification. This will be achieved by bioprinting a biomaterial laden with stem cells (e.g., but not limited to, mesenchymal stem cells, MSCs), or chondrocytes, into a porous bone-like structure and inducing cartilage formation. Endochondral ossification of these bone-like cartilage templates provides proper bone formation. Thus, the ex vivo-generated templates described herein serve as a bioresorbable, regenerative graft for bone defects as well as an in vitro platform for both bone pathology research and drug screening. As used herein, the terms bioresorbable and biodegradable mean that the material, once implanted into a host, will degrade. In addition, the versatile nature of the biofabrication platform used to generate the cartilage template allows for tailoring according to defect size in the case of bone repair as well as the tailoring of porosity, microstructure and cell density in the case of in vitro disease models.

The embodiments described provide precise spatio-temporal control over the structure and cell microenvironment of a porous cartilage scaffold. Other researchers have used 3D-printing to make nonporous cartilage scaffolds, and also with no temporal control over the incorporation or release of bioactive factors.

From a spatial standpoint, current methods of preparation of cell-laden cartilage templates do not provide control over the size, the shape, the mechanical stiffness, the loading distribution nor the structural integrity of the construct. The embodiments described provide a 3D-printer with a precision of 200 μm as well as a biomaterial with tunable rheological properties which allows for fine control over the shape, dimensions, integrity and stress distribution of the construct. It is noted that 3D-printed hydrogel structures are very different from molded structures. 3D-printing provides control over the structure of the cartilage template that is absent in molded structures. In particular, while the elastic moduli of printed vs. molded constructs are consistent, surprisingly, time-dependent mechanical properties (i.e. viscoelastic distribution of stress), porosity, and swelling properties vary significantly between the two (see Example 5).

From a temporal standpoint, current methods used to induce chondrogenesis in the construct are inefficient and cannot mimic the delivery sequence of various factors/cytokines needed for chondrogenesis in native cartilage. The embodiments described employ encapsulated protein-loaded microparticles into the 3D-printed template, which allows spatiotemporal control over signaling molecules. This further provides the control needed to mimic the cytokine delivery sequence found in native tissue.

All ranges referred to herein include all sub-ranges, integers, and fractions of integers, unless otherwise provided.

The terms “comprising,” “comprises,” “contains,” “containing,” “has,” “have,” “having,” “include,” includes,” “including”, and the like, are used interchangeably and indicate that the subject is open ended, unless otherwise noted.

The terms “consist,” “consists,” “consisting,” and the like, are used interchangeably and indicate that the subject is closed ended, unless otherwise noted.

Throughout this application, where compositions, components, methods, or steps are described as required in one or more embodiments, additional embodiments are contemplated and are disclosed hereby for fewer compositions, components, methods, or steps, and for fewer compositions, components, methods, or steps in addition to other compositions, components, methods, or steps. All compositions, components, methods, or steps provided herein may be combined with one or more of any of the other compositions, components, methods, or steps provided herein unless otherwise indicated.

The term “autologous” in reference to cells or tissue, unless otherwise noted, is intended to mean that the cell or tissue is obtained, directly or indirectly, from the same individual subject to which it is to be delivered. Unless otherwise noted, the term “autologous” includes cells or tissues derived from cells or tissues obtained, directly or in indirectly, from the same individual subject to which it is to be delivered.

The term “allogeneic” in reference to cells or tissue, unless otherwise noted, is intended to mean that the cell or tissue is obtained, directly or indirectly, from a different individual of the same species than the subject to which it is to be delivered. Unless otherwise noted, the term “allogeneic” includes cells or tissues derived from cells or tissues obtained, directly or in indirectly, from a different individual of the same species than the subject to which it is to be delivered.

The 3D porous cartilage templates described herein are made of biocompatible materials, meaning either synthetic or natural materials that interface with biological systems without inducing an undesirable immune response. Examples include polymers and hydrogels described herein and within the literature cited herein. The templates utilized herein, and production techniques, include those described in the Examples hereto, as well as the supporting References, all of which are incorporated herein by reference.

The 3D porous cartilage templates described herein comprise a network of interconnected rod elements and plate elements. Rod and plate elements are the basic elements of trabecular bone samples. For each rod or plate element, the cross-sectional area and thickness may vary along the length of the element. The plate- or rod-like geometry of the template structure can be calculated by reference to the Structure (or Structural) Model Index (SMI), described by Hildebrand and Ruegsegger, Journal of Microscopy, vol. 185(1) (2003). In SMI, a value of 0 is assigned to plates, 3 for rods, and 4 for solid spheres. The templates described herein may have a SMI between 0 and 3, exclusive of the endpoints which reflect pure plates or pure rods. A value of 1.5 reflects equal proportions of plate and rod elements. Greater plate elements relative to rod elements is associated with increased strength of mature bone tissue. However, porosity due to spaces formed between rods and plates is understood to have a stress-distributive function.

In further embodiments, SMI is between about 0.05 and about 1.2, inclusive of endpoints, or between about 0.05 and about 1, inclusive of endpoints, or in any range therein within 0.001, 0.01, or 0.05 increments thereof. The SMI may also be between about 0.1 and about 1, about 0.1 and about 0.9, about 0.1 and about 0.8, about 0.1 and about 0.7, about 0.1 and about 0.6, about 0.1 and about 0.5, about 0.1 and about 0.4, about 0.1 and about 0.3, and about 0.1 and about 0.2, inclusive of endpoints. Still further embodiments reflect SMIs between, about 0.2 and about 1, about 0.3 and about 1, about 0.4 and about 1, about 0.5 and about 1, about 0.6 and about 1, about 0.7 and about 1, about 0.8 and about 1, and about 0.9 and about 1, inclusive of endpoints.

The templates can also be described by other measures, including bone volume fraction (bone volume (BV)/total volume (TV)), trabecular thickness (Tb.Th), trabecular spacing (Tb.Sp), bone surface density (bone surface (BS)/total volume (TV)), and ellipsoid factor (EF). For each of these indices, values and ranges associated with healthy bone are known from in the art and are incorporated herein as embodiments of the claimed templates.

The porous cartilage templates may have a volume range of each plate element between about 4×10⁶ μm³ and about 30×10⁶ μm³, inclusive of endpoints. Still further, the volume may range from between about 5×10⁶ μm³ and about 25×10⁶ μm³, about 5×10⁶ μm³ and about 20×10⁶ μm³, about 10×10⁶ μm³ and about 25×10⁶ μm³, about 10×10⁶ μm³ and about 20×10⁶ μm³, and about 10×10⁶ μm³ and about 15×10⁶ μm³, inclusive, as well as integers and fractional values within these ranges.

The porous cartilage templates may have a volume range of each rod element between about 2×10⁶ μm³ and about 15×10⁶ μm³, inclusive of endpoints. Still further, the volume may range from between about 5×10⁶ μm³ and about 15×10⁶ μm³, about 2×10⁶ μm³ and about 10×10⁶ μm³, and about 5×10⁶ μm³ and about 10×10⁶ μm³, inclusive, as well as integers and fractional values within these ranges.

The thickness of plate elements may be between about 50 and about 200 μm, inclusive. Still further, the thickness may be between about 50 and about 150 μm, between about 100 and about 200 μm, between about 150 and about 200 μm, or about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195 or about 200 μm, as well as integers and fractional values within these ranges.

The thickness of rod elements may be between about 50 and about 110 μm, inclusive. Still further, the thickness may be between about 50 and about 100 μm, between about 50 and about 75 μm, between about 75 and about 100 μm, or about 50, about 55, about 60, about 65, about 70, about 75, about 80, about 85, about 90, about 95, about 100, about 105 or about 110 μm, as well as integers and fractional values within these ranges.

Each rod element may have a geometric tortuosity range between about 1 and about 2.5, inclusive. Geometric tortuosity of a sinuous line (rod) is defined as the ratio of the length of the line to the distance between the two ends of the line. In further embodiments, the geometric tortuosity may range between about 1 and about 2, about 1.5 and about 2.5, about 1.5 and about 2, or be any integer or fractional value thereof within these ranges, including about 1.1, about 1.2, about 1.3, about 1.4, about 1.5, about 1.6, about 1.7, about 1.8, about 1.9, about 2.0, about 2.1, about 2.2, about 2.3, about 2.4 or about 2.5.

The separation range between any two elements of the template may be between about 0.3 and about 1.7 mm, inclusive. In further embodiments, the range may be between about 0.5 and about 1.5 mm, inclusive, or any fractional value thereof within these ranges, including about 0.3, about 0.4, about 0.5, about 0.6, about 0.7, about 0.8, about 0.9, about 1.0, about 1.1, about 1.2, about 1.3, about 1.4, about 1.5, about 1.6 or about 1.7 mm.

The numeric density range for all elements in a template may be between about 0.5 and about 3 mm⁻¹, inclusive. In further embodiments, the range may be between about 0.5 mm⁻¹ and about 2.5 mm⁻¹, between about 1 mm⁻¹ and about 2.5 mm⁻¹, between about 0.5 mm⁻¹ and about 1 mm⁻¹, or between about 2 mm⁻¹ and about 2.5 mm⁻¹, inclusive, or any fractional value thereof within these ranges, including about 0.5, about 0.7, about 0.8, about 0.9, about 1.0, about 1.1, about 1.2, about 1.3, about 1.4, about 1.5, about 1.6, 1.7, about 1.8, about 1.9, about 2.0, about 2.1, about 2.2, about 2.3, about 2.4, about 2.5, about 2.6, about 2.7, about 2.8, about 2.9 or about 3.0 mm⁻¹.

Moreover, the numeric density range for plate elements within a template may be between about 1.1 and about 2.5 mm⁻¹, inclusive. In further embodiments, the range may be between about 1.1 mm⁻¹ and about 2 mm⁻¹, between about 1.5 and about 2 mm⁻¹, or between about 1.5 and about 2.5 mm⁻¹, inclusive, or any fractional value thereof within these ranges, including about 1.1, about 1.2, about 1.3, about 1.4, about 1.5, about 1.6, 1.7, about 1.8, about 1.9, about 2.0, about 2.1, about 2.2, about 2.3, about 2.4 or about 2.5 mm⁻¹.

The numeric density range for rod elements within a template may be between about 1.6 and about 2.6 mm⁻¹, inclusive. In further embodiments, the range may be between about 1.6 mm⁻¹ and about 2.5 mm⁻¹, between about 1.6 mm⁻¹ and about 2.0 mm⁻¹, between about 2.0 mm⁻¹ and about 2.5 mm⁻¹, or any fractional value thereof within these ranges, including about 1.6, about 1.7, about 1.8, about 1.9, about 2.0, about 2.1, about 2.2, about 2.3, about 2.4 or about 2.5 or about 2.6 mm⁻¹.

Still further, the rod-rod connectivity density of the template may be between about 0.5 and about 8 mm³, inclusive. In further embodiments, the range may be between about 0.5 and about 6 mm³, between about 2 and about 8 mm³, between about 2.5 and 7.5 mm³, or any fractional value thereof within these ranges, including about 0.5, about 0.6, about 0.7, about 0.8, about 0.9, about 1.0, about 1.1, about 1.2, about 1.3, about 1.4, about 1.5, about 1.6, 1.7, about 1.8, about 1.9, about 2.0, about 2.1, about 2.2, about 2.3, about 2.4, about 2.5, about 2.6, about 2.7, about 2.8, about 2.9 or about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9 or about 8.0 mm³.

The plate-plate connectivity density may be between about 2 and about 35 mm³, inclusive. In further embodiments, the range may be between about 5 and 30 mm³, between about 10 and about 25 mm³, or between about 10 and 20 mm³, or any fractional value thereof within these ranges, including about 2.0, about 2.1, about 2.2, about 2.3, about 2.4, about 2.5, about 2.6, about 2.7, about 2.8, about 2.9 or about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9 or about 8.0, about 8.1, about 8.2, about 8.3, about 8.4, about 8.5, about 8.6, about 8.7, about 8.8, about 8.9 or about 9.0, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34 or about 35 mm³.

The rod-rod connectivity density may be between about 3 and about 35 mm³, inclusive. In further embodiments, the range may be between about 5 and 30 mm³, between about 10 and about 25 mm³, or between about 10 and 20 mm³, or any fractional value thereof within these ranges, including about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9 or about 8.0, about 8.1, about 8.2, about 8.3, about 8.4, about 8.5, about 8.6, about 8.7, about 8.8, about 8.9 or about 9.0, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34 or about 35 mm³.

The porosity of the template may be between about 30% and about 90% inclusive. In further embodiments, the porosity is between about 35% and about 75%, between about 40% and about 60%, or any fractional value thereof within these ranges, including about 30%, about 35%, about 40%, about 45%, about 50%, about 55%, about 60%, about 65%, about 70%, about 75%, about 80%, about 85% or about 90%.

The surface-to-volume ratio of the template may be between about 5 and about 25 mm²/mm³, inclusive. In further embodiments, the range may be between about 5 and about 25, or about 10 and about 25, or about 5 and about 20, or about 10 and about 20 mm²/mm³, or any fractional value thereof within these ranges, including about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9 or about 8.0, about 8.1, about 8.2, about 8.3, about 8.4, about 8.5, about 8.6, about 8.7, about 8.8, about 8.9 or about 9.0, about 10, about 11, about 12, about 13, about 14, about 15, about 16, about 17, about 18, about 19, about 20, about 21, about 22, about 23, about 24 or about 25 mm²/mm³.

Also provided is the fabrication of a porous cartilage template with 3D-bioprinting, which can be used for the study of endochondral ossification, bone disease, or for the generation of tissue engineering constructs for the replacement of damaged tissue. This technique allows precise control over the structure of the cartilage template and temporal control over the incorporation and/or release of bioactive factors.

Using a 3D-bioprinter, a gelatin methacrylate hydrogel containing human mesenchymal stem cells (MSCs) may be extruded and light-crosslinked into a 3D structure designed beforehand using computer aided design (CAD). Other materials can be used as bioinks, including collagen, hyaluronic acid, alginate, among others, as well as other crosslinking methods such as physical or ionic crosslinking. Alternatively, drug- or protein-loaded microparticles or nanoparticles may be incorporated during printing to promote chondrogenesis. Cytokines and other biological factors may be loaded via encapsulation or bioconjugation techniques. Chondrogenesis and chondrocyte hypertrophy may be assessed over time using immunohistochemistry (bone sialoprotein, collagen I, II, and X) and gene expression analysis (Col1, Col2, ColX, MMP13, Cbfa-1, OC, Bsp, Pthlh, PthR1, Bmp2, Bmp4, Bmp7).

In various embodiments, the porous cartilage template, in some embodiments the hydrogel, may contain one or more bioactive agents, including but not limited to growth factors and drugs. The delivery of bioactive agents to the site of a bone defect may be advantageous in some circumstances depending on the condition of the patient and the injury. In various embodiments, the bioactive agent may be an RGDS peptide or cartilage oligomeric matrix protein (COMP).

### A Method of Promoting the Repair of a Bone Defect in a Patient

In another aspect, the invention provides a method of promoting the repair of a bone defect in a patient by preparing a porous cartilage template having a bone-mimicking internal structure, embedding a plurality of cells into the porous cartilage template, and implanting the porous cartilage template into bone defect in the patient, thereby promoting the repair of the bone defect.

In some embodiments, the bone defect is first stabilized through, by way of non-limiting example, emergency surgery to immobilize the bone defect by the insertion of one or more selected from the group consisting of: compression plates, rods, nails, Kirschner wires, and casts.

In various embodiments, the porous cartilage template is prepared by 3D-printing. Methods of 3D-printing and suitable printers are discussed above and in the examples, in particular examples 1, 5 and 6. In general, the various embodiments described above with respect to the porous cartilage template are suitable for use in the instant method, as are the templates produced by following the method for producing a porous cartilage template described below.

In some embodiments, the bone defect is imaged and the template is 3D-printed based on the imaging data acquired. Imaging the bone defect allows the template to be prepared at a size and in a shape that maximizes its therapeutic benefit, in various embodiments by approximating the bone structure that would be present at the site, absent the injury. Any imaging technique capable of visualizing bone with enough resolution to satisfactorily image the bone defect in order to facilitate 3D-printing may be used. In various embodiments, imaging data may be acquired by computed tomography (CT) scan or magnetic resonance imaging.

As discussed in above embodiments, the cartilage template includes a hydrogel containing a plurality of cells. In various embodiments, the plurality of cells includes mesenchymal stem cells. In some embodiments, the mesenchymal stem cells are harvested from the patient. In some embodiments, the plurality of cells comprises chondrocytes. In various embodiments, the mesenchymal stem cells are cultured to differentiate into chondrocytes.

In various embodiments, the 3D-printing and embedding steps are performed simultaneously. In various embodiments, the plurality of cells is contained in a hydrogel that is 3D-printed to form at least a portion of the porous cartilage template. In some embodiments, the hydrogel diffuses a porous network in the template and subsequently crosslinked, as further described below.

In some embodiments, the template is implanted into the bone defect of the patient without cells in the hydrogel. In these embodiments, blood vessels from the surrounding tissue will infiltrate the porous channels, bringing osteoprogenitor cells that turn the cartilage into bone (ossification).

In various embodiments, the porous cartilage template may be secured to the bone defect using any technique deemed appropriate by a person of skill in the art. In various embodiments, the porous cartilage template is secured in the bone defect by press fitting.

### Method of Preparing a Porous Cartilage Template

In another aspect, the invention provides a method of preparing a porous cartilage template for bone repair, by 3D-printing a porous network based on bone imaging data, the porous network comprising: a support component, a sacrificial component, and a plurality of pores; casting a cell-carrier component comprising a plurality of cells into the plurality of pores, evacuating the sacrificial component to form a network of passages among the support component and cell-carrier component; and culturing the plurality of cells of cells to form mature cartilage; thereby forming the porous cartilage template.

In various embodiments, the support component is a stiff network that is water insoluble and slow degrading. Although the support component fulfills a variety of functions, in various embodiments it may assist in defining the shape of the construct until implanted cells mature and form cartilage and/or the cartilage ossifies into bone. In various embodiments, the support component includes polycaprolactone.

The sacrificial component will preserve space for a network of pores that will permeate the finished template. In various embodiments, the sacrificial component is water soluble to promote ease of evacuation. In order to facilitate printing, in various embodiments the sacrificial component has a melting point similar to or the same as the material that forms the support component. In various embodiments, the sacrificial component has a melting point of about 65° C. In various embodiments, the sacrificial component is polyethylene glycol 20,000.

The plurality of pores is formed between the support component and the sacrificial component upon 3D-printing. The cell-carrier component fills or substantially fills the plurality of pores. In various embodiments, the cell-carrier component is a hydrogel. In some embodiments, the hydrogel includes gellan gum and/or gelatin. In some embodiments, the hydrogel includes 0.75% w/v gellan gum and 0.25% w/v gelatin.

In some embodiments, the cell carrier component diffuses into the plurality of pores in a liquid, uncrosslinked state. In some embodiments, the method includes a step of applying a chemical cross-linker to the cell-carrier component after it has entered the plurality of pores. In some embodiments, the cross-linker is calcium chloride.

In various embodiments, the sacrificial component is evacuated from the construct after or simultaneously with the entry of the cell-carrier component into the plurality of pores. In various, embodiments, the sacrificial component is evacuated by dissolution in aqueous solution. Without wishing to be limited by theory, evacuating the sacrificial component creates a network of passageways in the construct that leaves room for perfusion and infiltration by blood vessels from the patient after implantation of the completed template.

After the cell-carrier component enters the plurality of pores, the cells are cultured to develop mature cartilage. In some embodiments, the same liquid that dissolves the sacrificial component may maintain the plurality of cells. In some embodiments, the liquid may be media. In some embodiments, the media may be minimum essential medium eagle. In various embodiments, the media may contain various factors that control or encourage differentiation and/or development of the cells.

The embodiments described above further include that matter contained within the following examples, the claims, and any other component of the application.

### EXAMPLES

The invention is now described with reference to the following examples. These examples are provided for the purpose of illustration only and the invention should in no way be construed as being limited to these examples but rather should be construed to encompass any and all variations that become evident as a result of the teaching provided herein. The specific embodiments described in the Examples are intended to be embodiments of the invention.

### Example 1—Optimization of 3D Porous Cartilage Template Printing Using Human Mscs

Rationale.

The first step in developing a model of endochondral ossification is the generation of a porous cartilage template. While cartilage has been engineered for decades using human MSCs cultured on porous scaffolds, chondrocytes do not reside on porous structures in the body. Rather, they are encapsulated within dense ECM, even when this structure constitutes a macroscopically porous template like it does during endochondral ossification. For this reason, cartilage engineering is typically conducted by encapsulating the cells within a matrix that closely resembles the native ECM, such as a hydrogel. Hydrogels, 3D crosslinked polymer networks swollen with water, can be prepared from synthetic or naturally derived polymers. The stiffness and crosslinking density of the hydrogel matrix affects the development of cartilage tissue. However, the generation of a porous hydrogel structure in which chondrocytes are encapsulated within the struts of the construct has not been investigated. Therefore, this aim will focus on optimization of methods to generate such a porous structure, and the effects of various structural parameters on chondrocyte hypertrophy, the event that signals the start of endochondral ossification.

Experimental Design.

Gelatin was chosen as the hydrogel for printing because it is derived from collagen, the main component of cartilage, and it can be readily modified with standard bioconjugation techniques. In order to precisely control the mechanical properties of the hydrogel, which affects both the structural integrity of the printed construct and the chondrogenesis of encapsulated MSCs, a methacrylate group was introduced to the gelatin, allowing covalent crosslinking initiated by the addition of trace amounts of a photoinitiator activated by visible light. Human MSCs, obtained from a commercially available source, are mixed with the gelatin solution and extruded from a disposable syringe attached to a custom-designed 3D-printer developed by our close collaborators BIOBOTS™, Inc. (Philadelphia, Pa.), which controls movement in three dimensions according to user-generated CAD models. The gelatin content and degree of crosslinking are chosen in order to maximize chondrogenesis of MSCs. The extruding pressure and speed of printing are varied to optimize printing resolution, fidelity of the printed structure, and viability of encapsulated MSCs (Table 1).

TABLE 1
Experimental variables and outcomes

| Printing | | Structural | |
|---|---|---|---|
| Variables | Outcomes | Variables | Outcomes |
| Gelatin content | Structural | Solid gel | Chondrocyte |
| and crosslinking | integrity | | differentiation and |
| Printing speed | Fidelity of | Bone-like porous | hypertrophy |
| | structure and | structure | (immuno- |
| | printing | | histochemistry and |
| | resolution | | gene expression) |
| Extrusion | MSC | Cross-hatch | |
| pressure | viability | structure | |

Once control over printing is optimized, three different structures are printed to compare their effects on hypertrophy of chondrogenically differentiated MSCs: i) a solid hydrogel, which is most similar to native articular cartilage; ii) a porous bone-like structure modeled from CT scans of human cancellous bone; and iii) a porous cross-hatch structure designed to have similar overall porosity to native bone but with a different distribution, thereby separating the effects of actual structure from differences in mass transport. MSCs are printed within these structures and cultured for 1-5 weeks in chondrogenic media containing transforming growth factor-β1 (TGFβ1). Chondrogenesis and chondrocyte hypertrophy are assessed over time using immunohistochemistry (bone sialoprotein, collagen I, II, and X) and gene expression analysis (Col1, Col2, ColX, MMP13, Cbfa-1, OC, Bsp, Pthlh, PthR1, Bmp2, Bmp4, Bmp7).

Expected Outcomes and Alternative Strategies.

The structure of the construct may affect MSC chondrogenesis and chondrocyte hypertrophy. In particular, solid structures will support a stable cartilage phenotype, while porous structures will support chondrocyte hypertrophy. Design of cartilage may be performed consistent with documents. The choice of material or the process may be modified to optimize MSC viability and chondrogenesis. Structural signals alone can be sufficient to induce hypertrophy. Alternatively, the process is promoted through the withdrawal of TGFβ1 and the introduction of β-glycerophosphate and 1-thyroxin for the final 2 weeks of culture. Then, the effects of structure are investigated in an environment that is favorable for hypertrophy. The addition of soluble signals may override structural cues, so that differences in hypertrophy are observed in the different structures. Porous structures for the studies described herein are used, because an interconnected pore network is required for infiltration of blood vessels.

### Example 2—Effects of Dynamic Matrix Composition on Chondrocyte Hypertrophy

Rationale.

The composition of the ECM is extremely important in cartilage development. Many investigators have explored the effects of incorporating various ECM components into hydrogels, including glycosaminoglycans (GAGs) and different types of collagen. However, in normal cartilage development, the content of the ECM varies dramatically over time. For example, MSCs undergoing chondrogenic differentiation produce the ECM component fibronectin for about 10 days, and then it is downregulated. The importance of temporal control over this biochemical cue in MSC chondrogenesis was demonstrated when fibronectin fragments were released from synthetic hydrogels via a light-activated degradation strategy according to the temporal profile observed in development. Chondrogenic differentiation of encapsulated MSCs was enhanced compared to hydrogels containing persistent levels of fibronectin. In order to examine the effects of signals that change over time, an important aspect of normal development, sophisticated drug delivery techniques must be employed. Applicant previously developed a technique to control both the conjugation and the release of ECM components from hydrogels at different times. The method is based on the strong and specific binding affinity between analogs of biotin with streptavidin. By varying the association and dissociation properties of the affinity pairs, ECM components can be introduced and released at pre-determined rates and time according to the kinetics of affinity-based drug delivery systems.

Experimental Design, Expected Outcomes, and Alternative Strategies.

To demonstrate the use of our novel technology to temporally control ECM composition, the fibronectin fragment RGDS are incorporated into the gel structure via biotin analog-streptavidin interactions so that it is slowly released over 10 days in vitro, using our previously described methods. The release profile is confirmed by measuring the daily release of a fluorescently conjugated version of RGDS. As a control, hydrogels with persistent levels of RGDS were prepared through covalent incorporation during crosslinking (Table 2). The controlled release of RGDS will enhance chondrogenic differentiation of MSCs compared to its persistent presence, as has been previously shown.

TABLE 2
Experiments

| ECM Experiments | Outcomes |
|---|---|
| Early release vs. persistence of | MSC chondrogenesis |
| RGDS | |
| Persistence vs. late | Cartilage formation and chondrocyte |
| incorporation of COMP | hypertrophy |

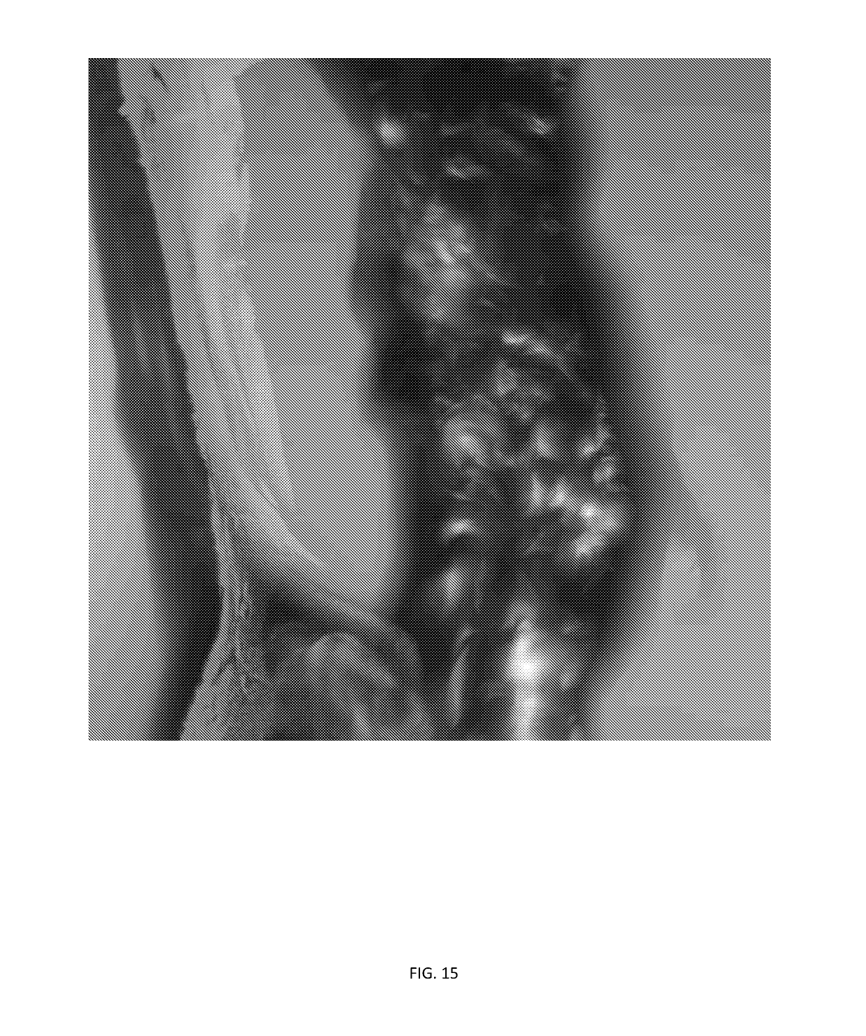

The effects of the delayed incorporation of cartilage oligomeric matrix protein (COMP), which is known to be involved in later stages of cartilage development, endochondral ossification, and the development of osteoarthritis, are investigated. The effects of COMP on chondrocyte hypertrophy are elucidated. COMP will be incorporated into the structure of the hydrogel matrix around 2 weeks after the start of culture, according to the temporal profile observed in normal cartilage development. COMP can be covalently incorporated at this point using methacrylation and light-activated conjugation, or it can be transiently incorporated using biotin analog-streptavidin affinity interactions (FIG. 15), as described above, which would allow its conjugation and subsequent release. As a control for the delayed introduction of COMP, it is incorporated covalently during construct fabrication. The incorporation of COMP and its release over time is confirmed using immunohistochemical analysis of the constructs without encapsulated MSCs. The delayed introduction of COMP will cause increased cartilage tissue formation and chondrocyte hypertrophy compared to its introduction at the start of culture. Alternatively, these methods are used to study the effects of collagen X, which is secreted by hypertrophic chondrocytes but has not been thoroughly investigated for its direct effects on pre-hypertrophic chondrocytes.

### Example 3—Differences Between Bone-Like Structure (Described Embodiments), a Lattice Structure (Control 1) and a Non-Porous Structure (Control 2)

A. Percent porosity and pose size of bone-like and lattice structures is calculated from CAD designs. Similar measurement values between the two structures removes porosity as a variable in experiments comparing structure.

B. The stress distribution of all three structures is evaluated using finite element analysis. The described embodiments have preferred stress distribution characteristics.

C. Chondrocyte pellet-laden gels are printed into all three structures and cultured for three days. Live/dead staining of all constructs is performed to assess cell viability. Glycosaminoglycans (GAGs) staining of all constructs after three days of culture is performed to evaluate cartilage tissue formation.

D. The culture described in (C) is extended to three weeks after extrusion of all structures. The constructs are stained for collagen and GAGs to evaluate cartilage tissue formation.

E. Bulk mechanical testing of all constructs is performed at day 0 and day 21 of culture. Elastic modulus, creep behavior, and stress relaxation behavior under unconfined compression is evaluated.

### Example 4—Effect of 3D-Bioprinting Vs. Molding

A. Gelatin methacrylate (GelMA) was synthesized using previously described methods. Briefly, a 10% w/v solution was prepared by dissolving gelatin (Type A, 300 bloom, porcine skin, Sigma Aldrich) in phosphate buffered saline (PBS) at approximately 60° C. Following complete dissolution, the solution temperature was maintained at 50° C. and 0.14 mL methacrylic anhydride was added for each gram of dissolved gelatin. The methacrylation reaction was allowed to proceed for 4 hours at 50° C. under vigorous stirring. PBS warmed to 40° C. was added to obtain a GelMA concentration of 4.5% w/v, and then ice-cold acetone was added at a volumetric GelMA solution-to-acetone ratio of 1:4, allowing the GelMA to precipitate overnight. The precipitate was dried and dissolved in PBS at a concentration of 10% w/v by heating to approximately 50° C. Following vacuum filtration through a 0.22 μm filter (polyethersulfone membrane, FISHER SCIENTIFIC™), the solution was dialyzed (Slide-A-Lyzer G2 Dialysis Cassettes, gamma-irradiated, 10K molecular weight cutoff, FISHER SCIENTIFIC™) for 3 days against deionized water with dialysis media change twice a day. The GelMA solution was finally lyophilized for four days and stored at −20° C.

B. Hydrogel fabrication by bioprinting and molding. Lithium phenyl-2,4,6-trimethylbenzoylphosphinate (LAP), a cytocompatible photoinitiator activated by visible light at a wavelength of 405 nm, was used to initiate photocrosslinking of GelMA. Hydrogels were prepared by dissolving synthesized GelMA at 10-20 w/v % in PBS along with LAP (BIOBOTS™) at 0.25% or 0.5%, as indicated. The employed bioprinting system was a BIOBOTS™ Beta pneumatic extruder, which is equipped with an extrusion pressure range of 0-140 psi and violet light irradiation capability at a wavelength of 405 nm. Prepared hydrogel formulations were loaded in a 10 mL syringe (BD) fitted with a 27 gauge nozzle (200 μm inner diameter, JENSEN GLOBAL DISPENSING) for extrusion. Computer models for the constructs (either lines or cylinders) were designed using CREO PARAMETRIC™ 3.0 and imported into Repetier-Host software, where printing speed was set prior to extrusion. Molded cylinders were prepared by letting GelMA solutions gelate at solution depths matching that of extruded counterparts and irradiating the solutions under violet light. Biopsy punches (FISHER SCIENTIFIC™) were subsequently used to obtain molded cylinders with the exact same dimensions as extruded cylinders. Petri dishes were used as extrusion and molding substrates for extrusion pressure testing, line width evaluation, all mechanical testing and the swelling study, while glass slides were used for the microstructural analysis of constructs with optical microscopy. For both extruded and molded hydrogels, violet light irradiation for photocrosslinking was set to 10 minutes.

C. Microscopy and image analysis. Single-layer extruded lines were imaged using an EVOS microscope (transmitted light, phase contrast, 4× magnification). The total area of each line (obtained from ImageJ) was divided by the total length to obtain the average line width. Using a Zeiss AxioObserver Z1 microscope (phase contrast, 10× magnification), optical micrographs of extruded and molded cylinders (15 mm diameter, 1 mm height) were taken to assess differences in microstructure. The focal plane of the micrographs was set to the surface of the glass, i.e. the plane of contact between the hydrogel and the glass substrate.

D. Mechanical testing. For mechanical testing, cylinders (5 mm diameter, 1.4 mm height) were placed on the compression platens of a BOSE ELECTROFORCE 3220™ in an unconfined setup, immersed in PBS, and preloaded with a compressive stress of 2.5 kPa prior to each test. To measure Young's modulus, a strain of 33% was reached linearly over 120 seconds. Linear regression was performed on the obtained stress-strain data over the initial 7% of strain to obtain the slope of the initial linear portion of the stress-strain curve (Young's modulus). For creep testing, a 5 kPa stress was applied to the cylinders for 7 minutes (creep portion) followed by removal of stress for 7 minutes (recovery portion). Exponential fitting of the creep and recovery portions of the data was performed using equations (1) and (2) respectively, where s represents strain. Note that t corresponds to elapsed time from the moment at which a stress of 5 kPa was reached for equation (1) and the moment at which a stress of 0 Pa was reached for equation (2). a_{creep} and a_{recovery} correspond to the changes in strain caused by creep and recovery respectively while b_{creep} and b_{recovery} correspond to the equilibrium strain values of the creep and recovery portions respectively. r is a time constant that corresponds roughly to the amount of time it takes for the strain to reach around 37% of its final value (1/e).

$$\begin{matrix}ɛ ( t ) = {a}_{creep} exp ( - \frac{t}{{τ}_{creep}} ) + {b}_{creep} & ( 1 ) \\ ɛ ( t ) = - {a}_{recovery} exp ( - \frac{t}{{τ}_{creep}} ) + {b}_{recovery} & ( 2 )\end{matrix}$$

From the fitting, time-dependent mechanical behavior was quantified using four properties, namely extent of creep, average creep rate, extent of recovery and average recovery rate. The extent of creep is the total change in strain caused by creep while the extent of recovery is the percentage of this strain change that is recovered during unloading. Average creep and recovery rates correspond to the average rates of change in strain over the initial 99% of creep and recovery respectively.

E. Swelling kinetics. A swelling kinetics study was performed to assess the impact of microstructural differences on fluid flow. Molded and extruded cylinders (5 mm diameter, 1.4 mm height) were fully dried before they were each immersed in 10 mL PBS. Cylinders were subsequently weighed at multiple time points over 5 days of swelling. Swelling percentage was calculated using equation (3), where Mₜ corresponds to the hydrogel mass at time t and M₀ corresponds to the initial weight of the dried polymer prior to immersion in PBS.

$$Swelling percentage = \frac{{M}_{t} - {M}_{0}}{{M}_{0}} \times 100 \% \qquad ( 3 )$$

F. Statistical analysis. Two way-ANOVA with post-hoc Tukey analysis was performed to determine statistical significance between groups in the line width and Young's modulus data. Two-tailed t-tests were performed to compare Young's moduli and creep parameters between extruded and molded cylinders. A two-tailed t-test with Holm-Sidak correction for multiple comparisons was performed on extruded and molded groups in the swelling percentage data. All data are shown as mean±SEM, with n=8 for line thickness data and n=6 for all mechanical testing and swelling data. A p-value of less than 0.05 was considered statistically significant.

### Results

i. Combinatorial Effects of Extrusion Parameters and Biomaterial Composition on Construct Quality and Resolution.

Qualitative characterization of extruded lines revealed the existence of an optimal extruding pressure at each GelMA concentration investigated. For each GelMA concentration, extrusion skips were observed at pressures below the optimal range while unevenly excessive outpour was observed above that range. At 10%, 15%, and 20% w/v GelMA, 60 psi, 80 psi, and 100 psi, respectively, resulted in non-continuous flow, uneven thickness, and beads instead of lines. At 10% and 15% w/v GelMA, 100 psi and 120 psi, respectively, resulted in excessive outpour, uneven thickness, and large chunks in lines. Extrusion pressures of 80 psi, 110-110 psi, and 130 psi for 10%, 15%, and 20% w/v GelMA, respectively, were found to be optimal pairings—optimal pressure for continuous flow and constant thickness.

The impact of printing speed on line resolution was assessed by extruding lines at travel feed rates of 4 mm/sec, 8 mm/sec and 12 mm/sec. As expected, increasing the travel feed rate resulted in a significant decrease in line width, corresponding to an increase in resolution. Interestingly, increasing the GelMA concentration from 10% to 20% w/v also resulted in a small but significant decrease in line width (two way ANOVA, p≤0.05).

ii. Impact of Extrusion Process on Bulk Mechanical Properties

As expected, increasing the GelMA concentration from 10% to 15% and 20% resulted in an increase in the Young's modulus of molded cylinders. The concentration of the photoinitiator (LAP) had no effect on hydrogel elastic behavior.

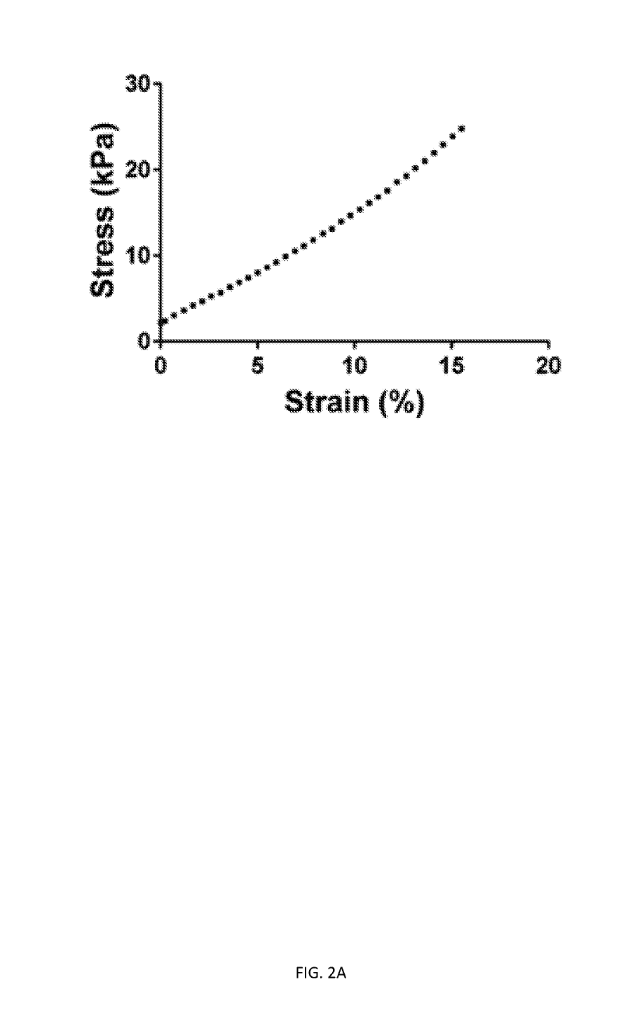

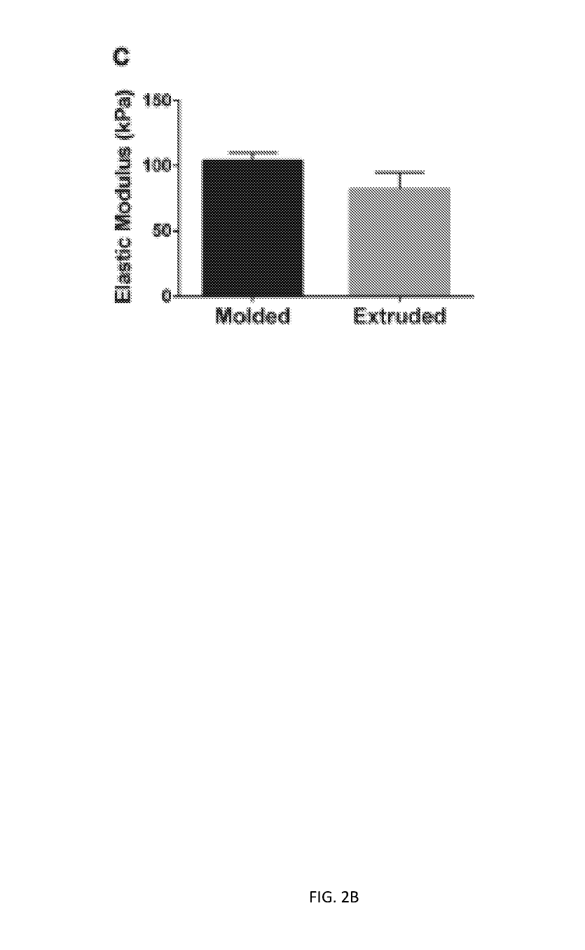

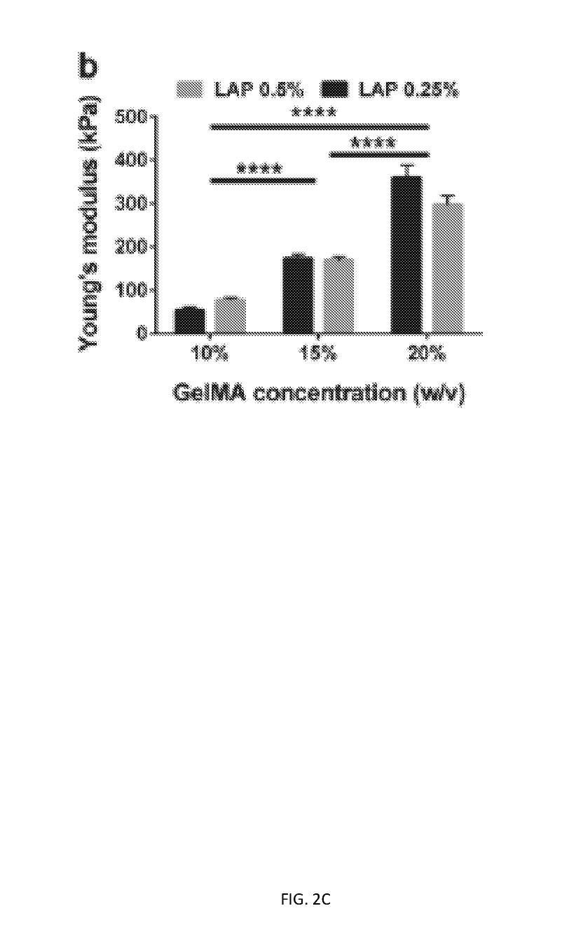

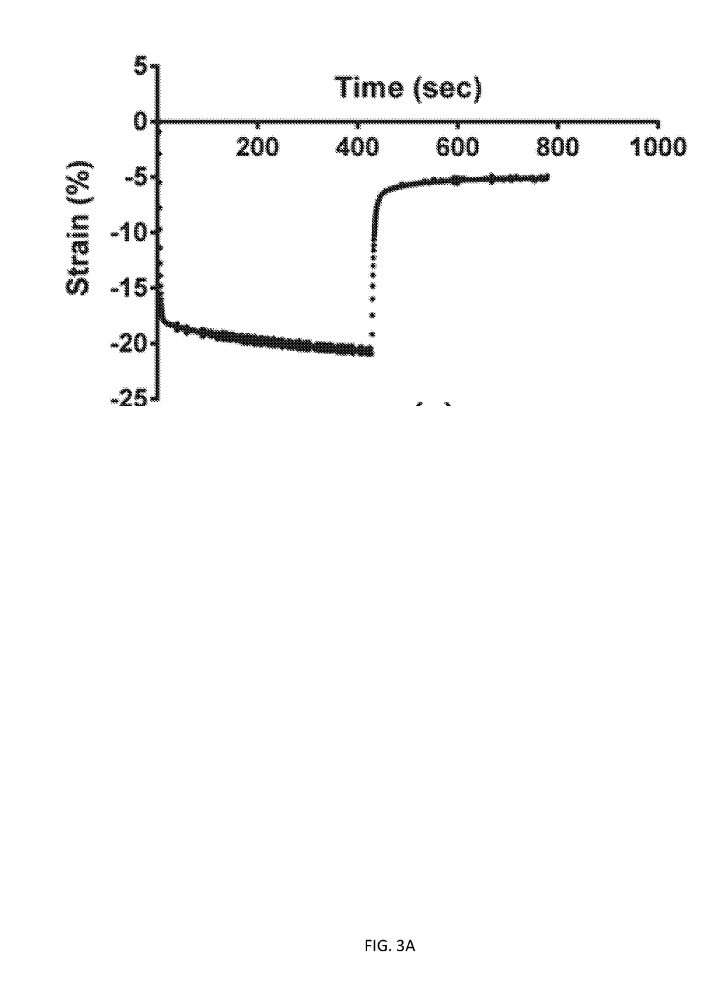

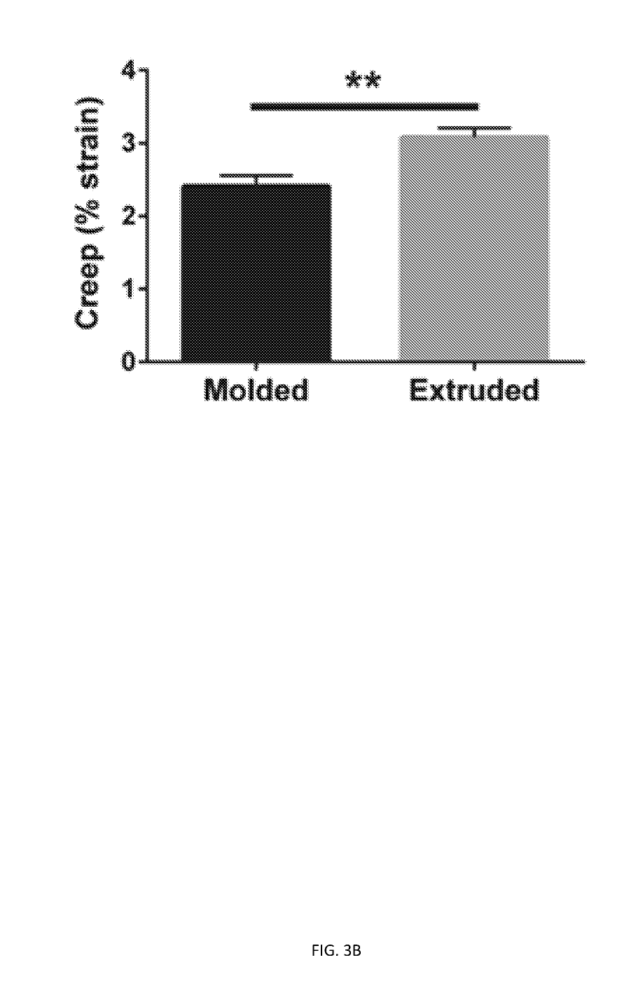

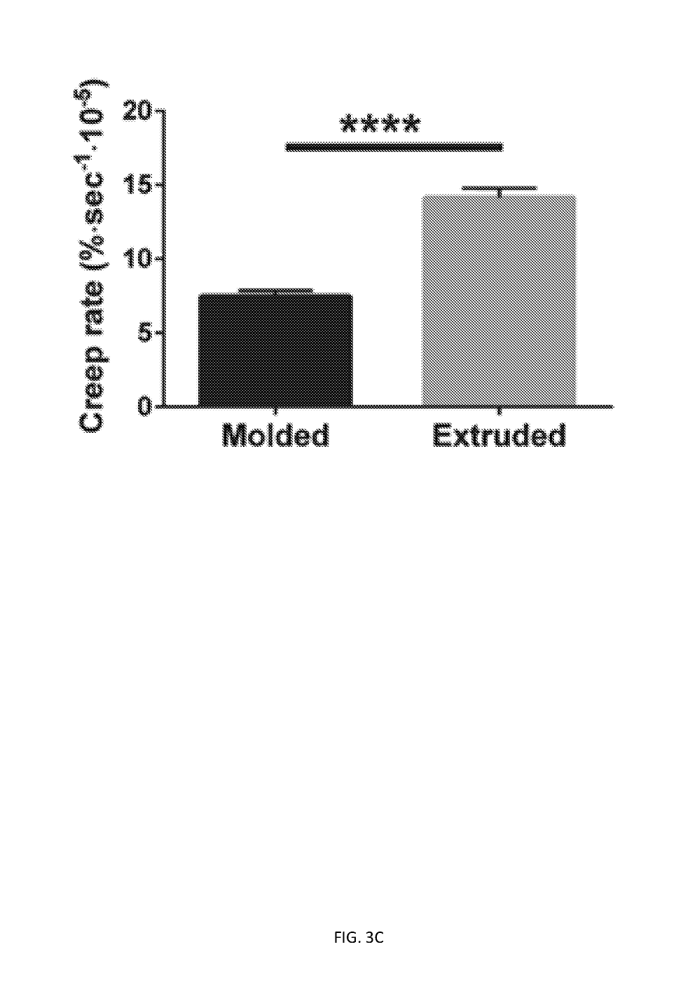

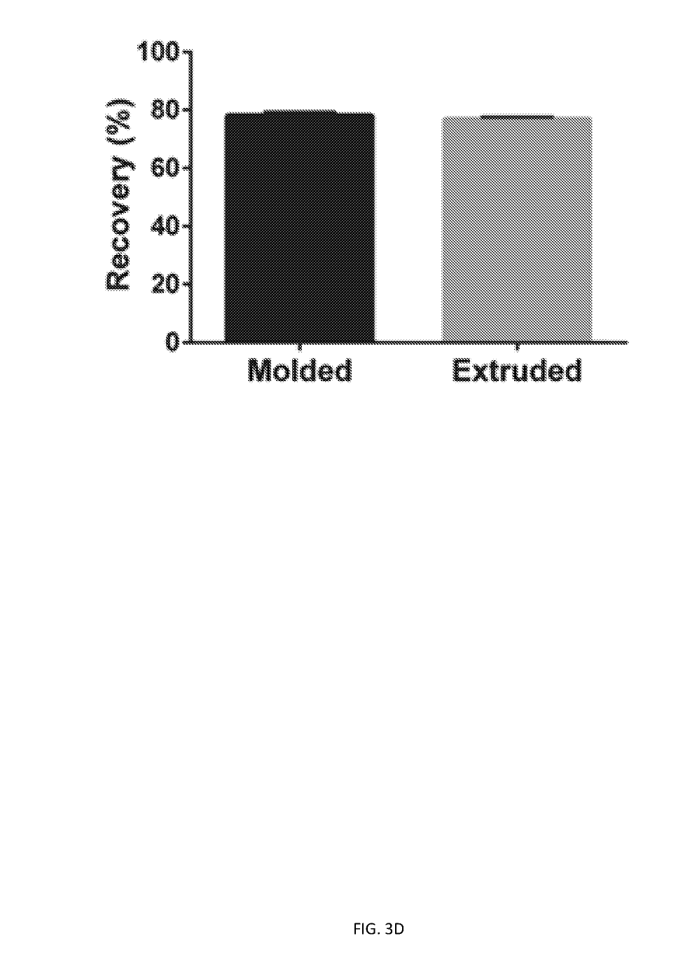

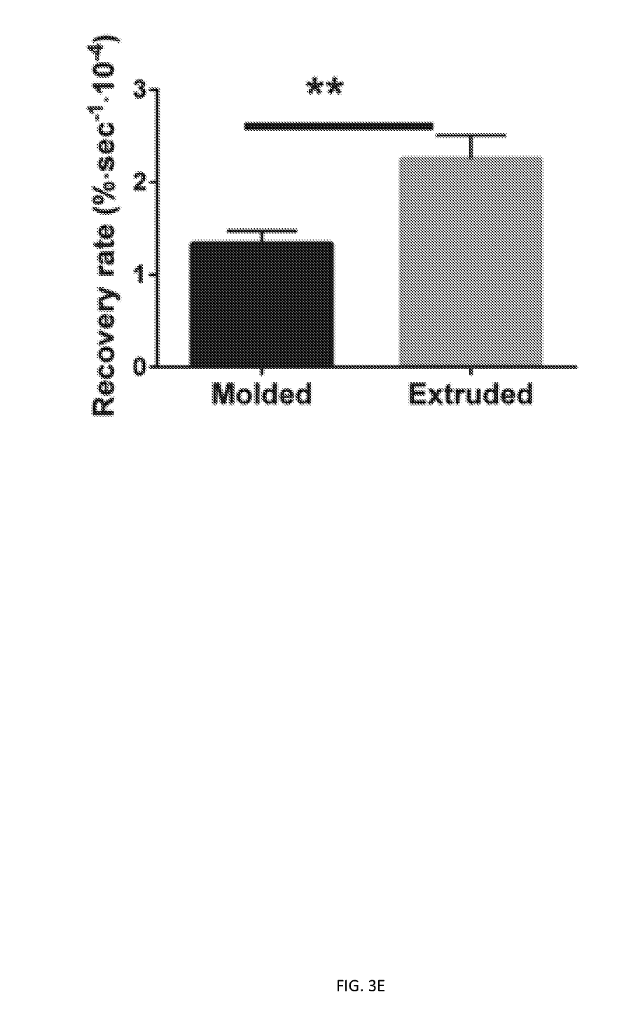

The impact of extrusion on Young's modulus was assessed by comparing molded and extruded hydrogel cylinders prepared with 15% GelMA and 0.25% LAP. Surprisingly, while no differences were observed in Young's modulus between molded and extruded cylinders (FIG. 2B), extruded constructs exhibited increased extents (FIG. 3B) and rates of creep (FIG. 3C) compared to molded constructs. Moreover, while the extent of recovery from creep was not different between extruded and molded constructs (FIG. 3D), the rate of recovery from creep was higher for extruded constructs (FIG. 3E). These results indicate that the extrusion process did not affect bulk elastic behavior (Young's modulus), but time-dependent mechanical behavior was affected.

iii. Impact of Extrusion Process on Microstructure and Swelling Properties

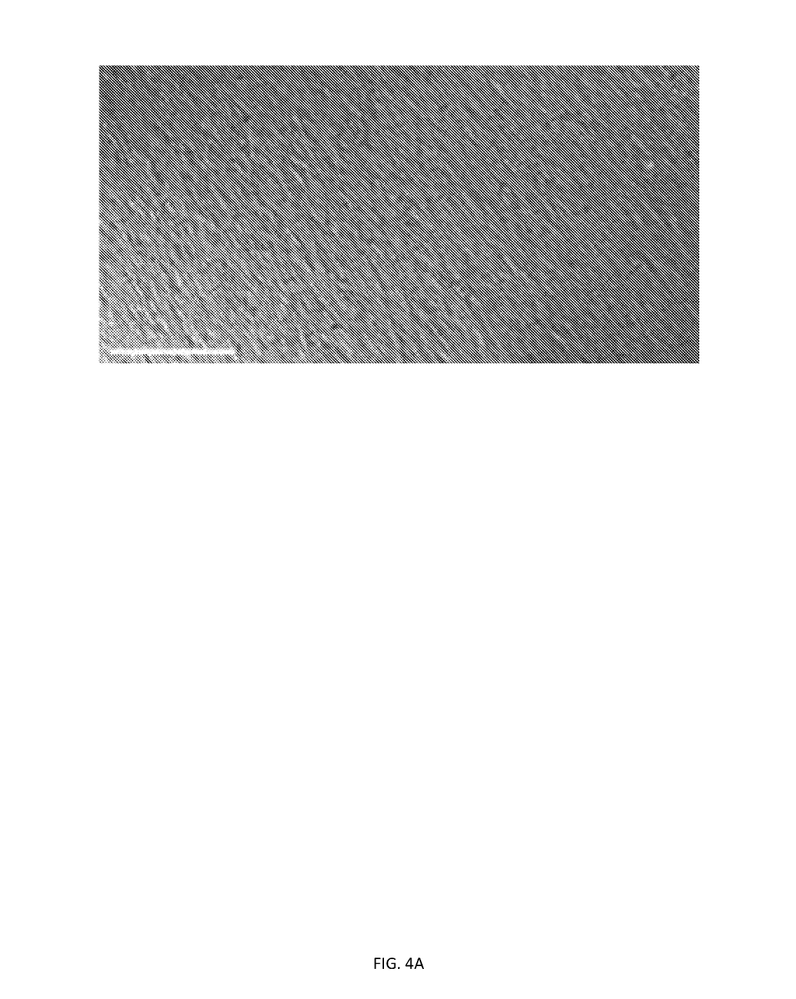

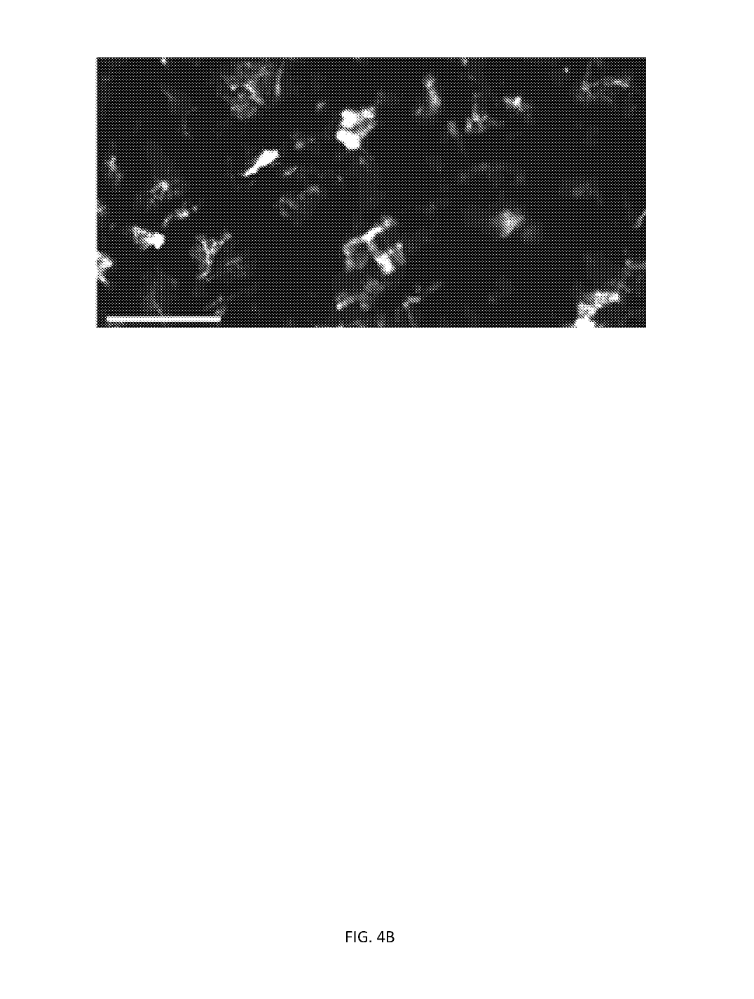

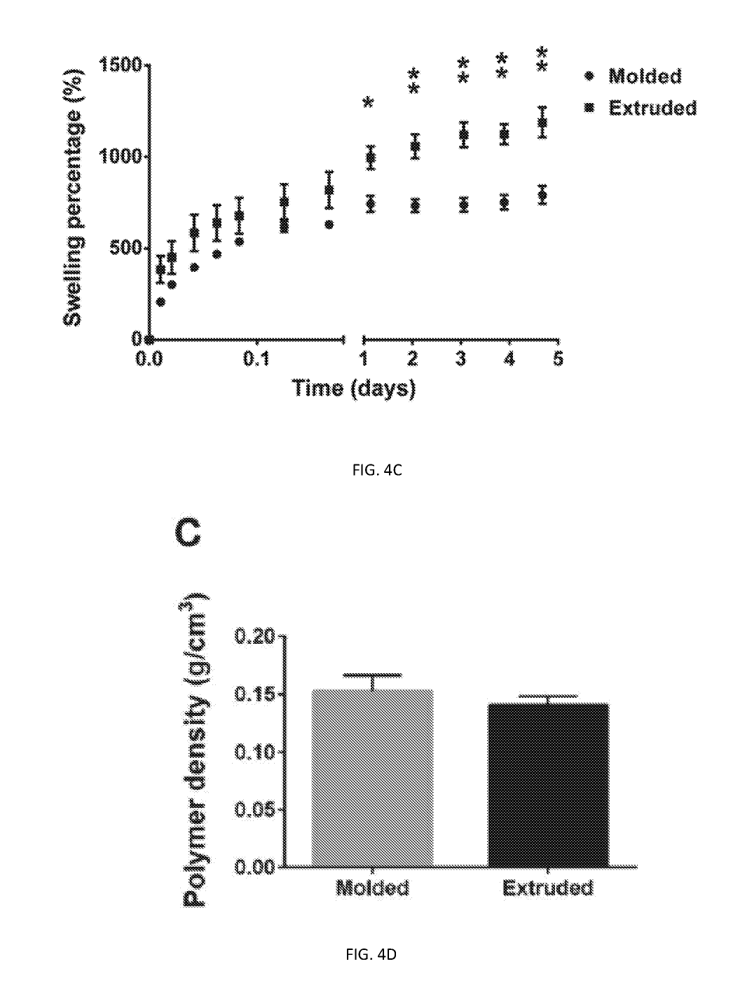

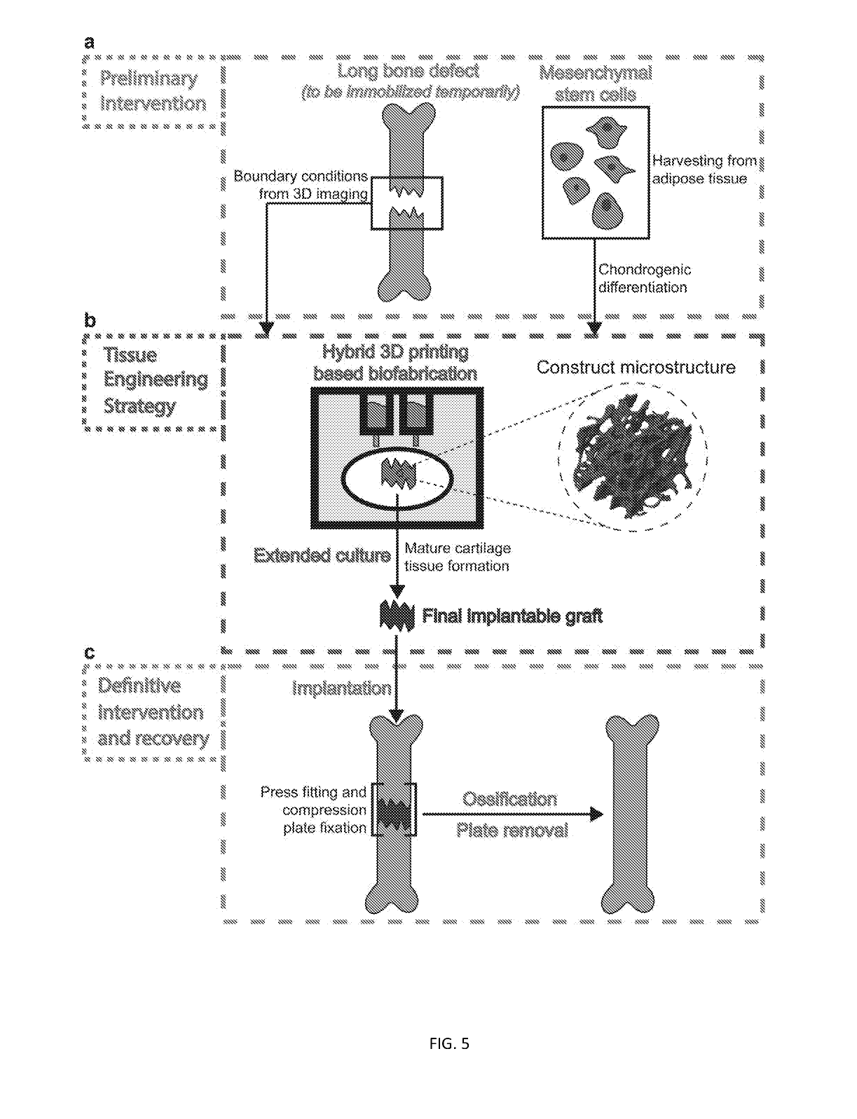

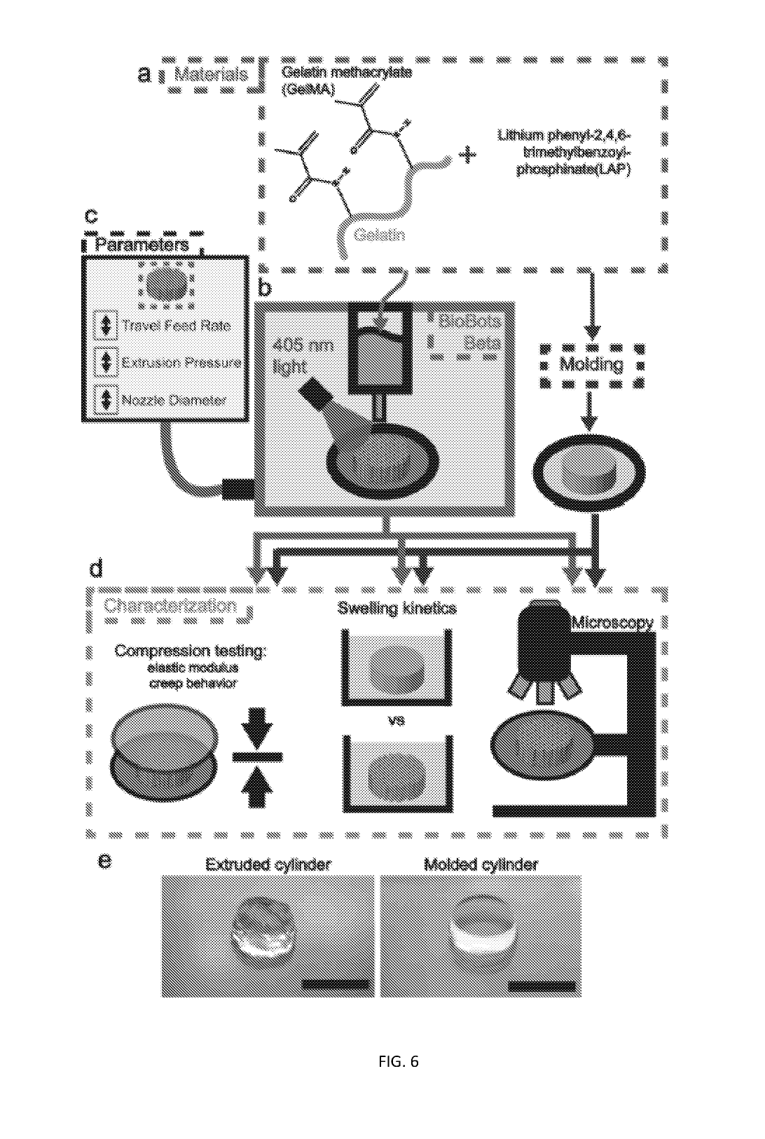

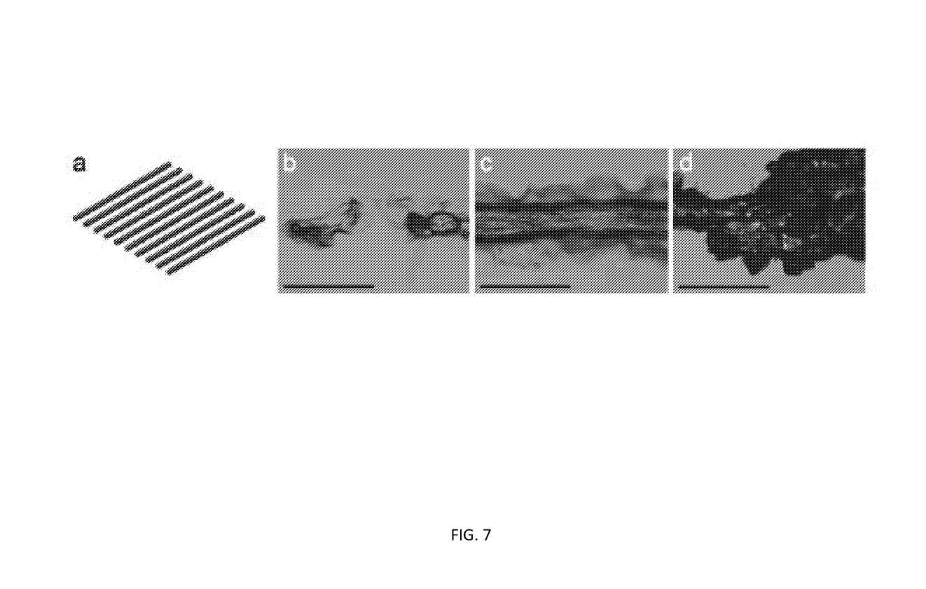

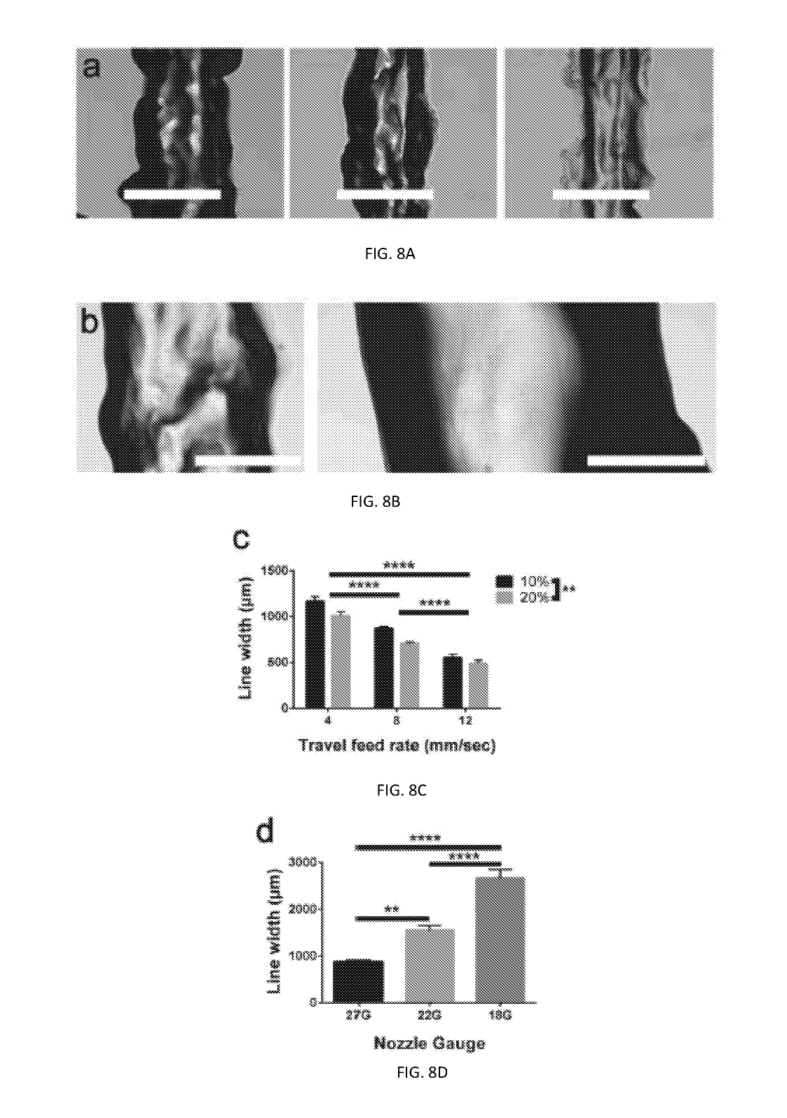

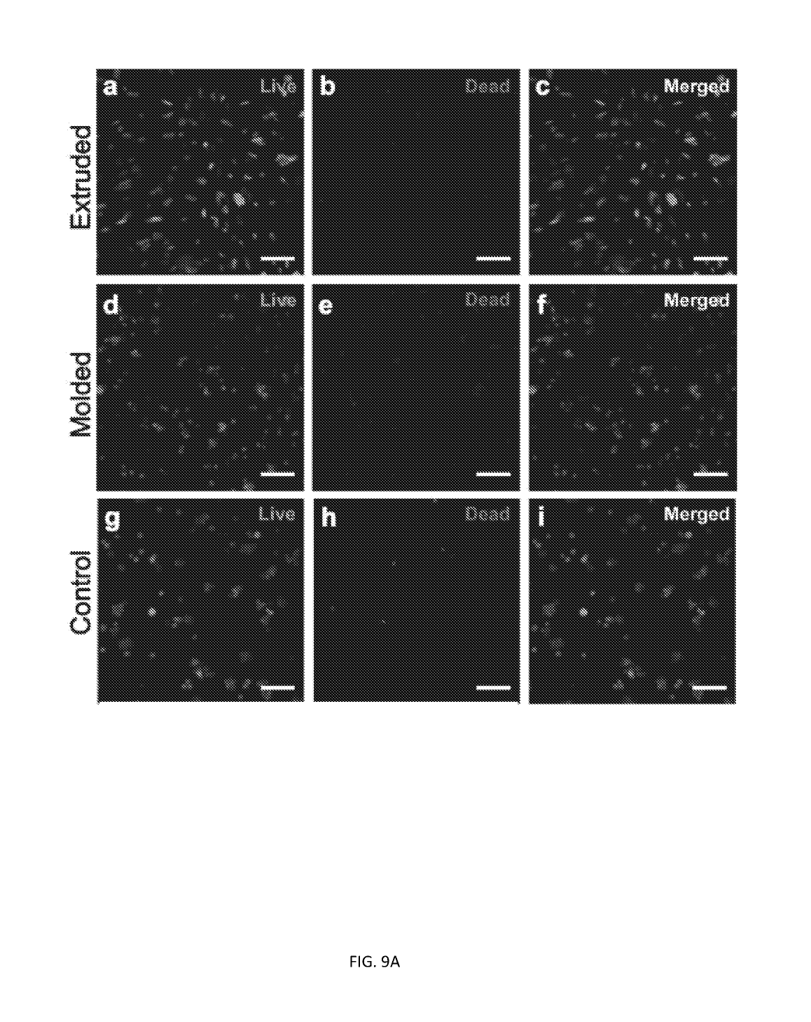

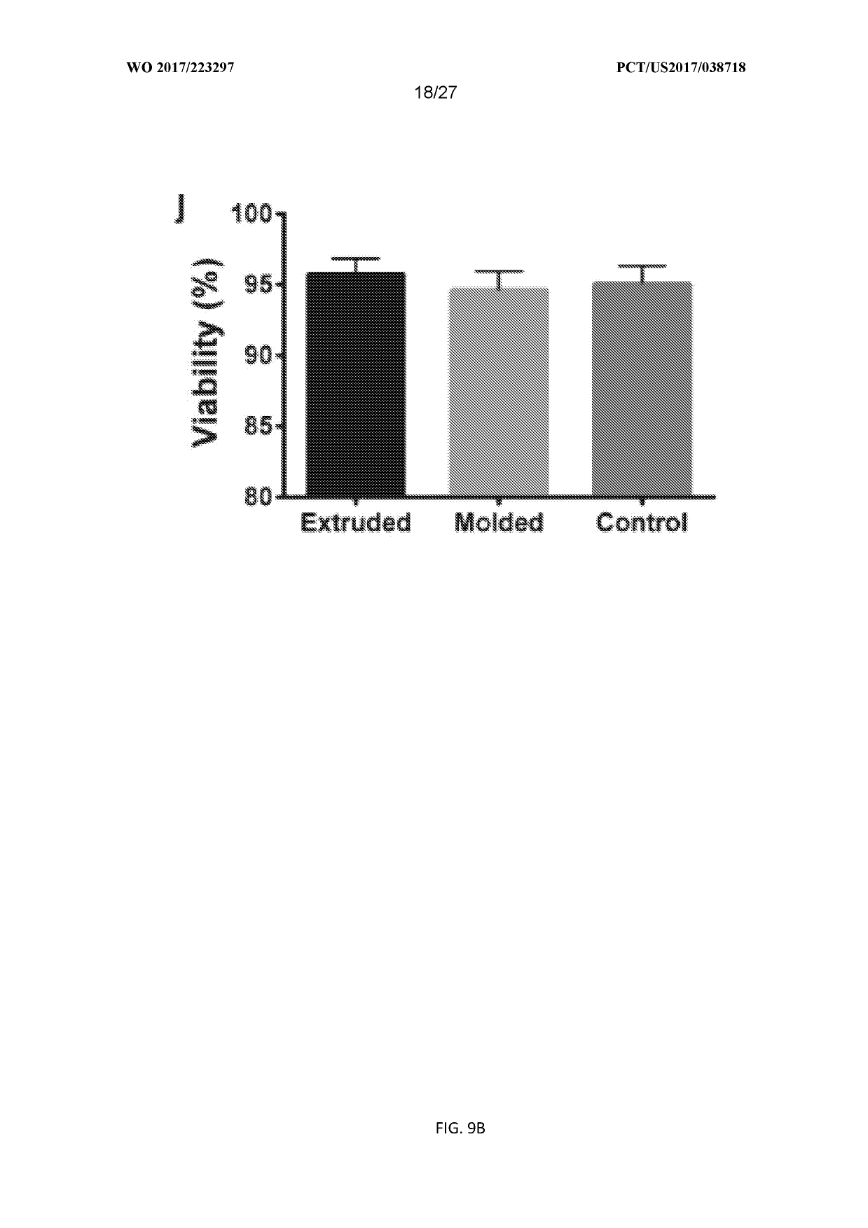

To investigate the mechanism behind the observed differences in time-dependent mechanical properties, molded and extruded cylinders were imaged under phase contrast microscopy. Molded constructs were characterized by uniform light transmission through the hydrogel (FIG. 4A) while extruded constructs were characterized by a variegated microstructure with extensive refraction caused by the presence of multiple discontinuities (FIG. 4B). As shown in FIG. 4C, differences in swelling behavior between extruded and molded constructs were apparent after 1 day, with extruded constructs exhibiting both faster and more extensive swelling compared to molded counterparts.

### Example 5

### Design Implementation: Criteria, Constraints and Envisioned Strategy.

In light of the proposed patient intervention strategy and the results from the previous examples, a number of criteria and constraints have been laid out to guide the development of the construct fabrication method:

Targeted defect types: As previously discussed, given that most cases of bone trauma, cancer and infection target long bones and that the endochondral ossification process is both endogenous to long bones during development and more widely studied in long bones in the context of native repair, templates will be targeted to critical size non-union defects involving long bones. In addition, it's important to note that non-union fractures require different intervention strategies depending on whether they're located at the midsection (diaphysis/metaphysis) or the distal/proximal ends (epiphysis/physis) because the two regions exhibit different compositional, geometric and mechanical properties. Therefore, since over 70% of long bone fractures occur in the diaphysis or metaphysis region and fractures along the midsection are often the gravest as they may break the skin and lead to infection, we've more specifically focused the target region of the proposed constructs to the midsection (diaphysis/metaphysis) of long bones.

Bulk size and shape: As previously mentioned, critical-size defects, which are not capable of being repaired natively, have lengths of more than 2.5 to 3 times the diameter of the affected bone and typically correspond to a volume range of 10-50 cm³. The fabrication method must therefore accommodate any bulk shape and size requirements within this volume range. This will be ensured by the scalability and shape conformation capabilities of additive manufacturing.

Surgical fixation: Taking into account that the generated constructs would be surgically affixed to the site of injury using press fitting and compression plate fixation, both of which are established scaffold fixation methods, the templates must withstand the press-fit strain needed for adequate fastening between the two separated bone segments. During press fitting, the prevention of implant sliding or loosening is ensured by the material and morphological properties at the implant surface as well as the strain experienced by the implant as a result of compression plate fixation. Since hydrogels lack the material and frictional properties to ensure press fitting regardless of the applied strain, a reinforcing network is required for the proposed surgical fixation method. This reinforcing network must be strong enough to withstand the press fit strain as well as any additional strain which may be the result of micromotions typically observed in bone fixation plates. To that end, the generated constructs must not fracture before a compressive strain of 1%, which is sufficiently large to account for the applied press fit strain as well as fixation plate micromotions.

Elastic modulus: Considering the importance of mechanotransduction in the ossification process, the bulk elastic behavior of the fabricated constructs should be around that of native cartilage-like callous tissue in the initial stages of healing. The range of elastic moduli reported for both native hyaline cartilage and early soft callus tissue is 1-5 MPa. Within three weeks of healing, the elastic modulus of the callus region is estimated to increase to 50 MPa. Since the hydrogel scaffolds to be fabricated will be reinforced with stiff networks in order to ensure the possibility of press fitting, it is expected that their bulk elastic modulus will exceed the 5 MPa upper limit of native early soft callus tissue. However, this modulus must not be so great that stress shielding occurs, preventing the mechanotransduction of encapsulated cells. An indicative point at which stress shielding becomes significant may be the appearance of woven bone, which is the earliest and most disorganized type of bone tissue formed during endochondral ossification prior to trabecular bone formation. Accordingly, the upper limit for the elastic modulus of the templates has been set to the lowest reported values for the elastic modulus of woven bone, i.e. around 30 MPa. Thus, the bulk elastic modulus of the generated constructs must lie between 5 and 30 MPa.

Printing precision and consistency: The use of additive manufacturing as part of the envisioned fabrication strategy is intended to ensure that complementary networks with varying architectures and dimensions are concurrently formed in each multi-material construct with accuracy over space and uniformity over time. This capability guarantees that different computer-generated architectures will lead to the formation of geometrically distinct experimental groups of constructs. Previous studies characterizing traditional 3D-printing methods hold that geometric measurements with relative standard deviations smaller than 20% are indicative of adequate reproducibility. Accordingly, to confirm that the employed additive manufacturing platform is able to attain the level of precision and consistency needed for adequate fidelity, the widths measured from any given strut element for a specific construct architecture must (1) not have a mean which deviates by more than 20% of the intended value (precision) and (2) not have a relative standard deviation of more than 20% (consistency).

Construct parameter modulation: As previously discussed, the capacity for parameter modulation as a requirement for the biofabrication platform in order to be able to conduct comprehensive studies with the generated constructs and to be able to tailor constructs on a case-by-case basis has been established. Given that porosity constitutes the construct property which mediates both mechanical and fluid flow behavior, it would be reasonable to select it as the primary metric for tailorability. For comparison, the porosity of trabecular bone typically varies between 70% and 90%, which amounts to a porosity range of 20%. Similarly, the devised biofabrication method must be able to generate constructs at various porosities over a min-max range which exceeds 20%. As an added criterion, significant differences in mechanical and/or swelling behavior must be observed depending on the porosity of the constructs generated.

Stiff material content: As a crucial component of endochondral ossification, vascularization is another important consideration in the design of the biofabrication platform. Of note in the context of this design is the fact that the reinforcing stiff network is expected to resorb in the span of weeks to months: the stiff material thus amounts to volume inaccessible to vasculature. Indeed, bloods vessels would only be able to invade through the porous network and, to a lesser extent, through remodeled areas of the hydrogel (which resorbs faster than the stiff material). Accordingly, a maximal threshold must be set for stiff material content within the constructs. In native settings, the lowest porosities recorded for trabecular bone is 30%, which corresponds to a maximal bone content of 70%. Hence, the maximal volumetric content of stiff material in the generated scaffolds has been set to be 70%.

Swelling: From the results in previous examples, it was found that the greatest swelling percentage recorded for a molded construct at the final time point (5 days) is 962%. Thus, to confirm the prevention of any excessive swelling deformation by the hydrogel component of the generated constructs, we've set the maximal limit for the swelling percentage of hydrogels within the generated constructs to 962% until day 5.

### Design Strategy.

In order to retain the spatial control that 3D-printing provides while barring the use of filament-based hydrogel extrusion, a fabrication technique which couples hybrid construct printing with hydrogel casting and sacrificial pore formation has been devised. More expressly, printing a two-component porous construct using stiff, thermoplastic materials through melt extrusion is envisioned. The hydrogel material (i.e. the cell-carrier component of the templates) is then cast into the porous network of the hybrid construct. Subsequently, one of the two stiff, thermoplastic components (i.e. the sacrificial network) of the construct is evacuated away, creating a secondary pore network for vascularization and nutrient supply. Thus, with this strategy, it is possible to accurately shape the architectures of the stiff network, the hydrogel network and the pore network without having to riddle the hydrogel with interstices by extrusion.

### Experimental Design, Fabrication Strategy and Material Selection.

Material selections were made to accommodate the devised fabrication strategy:

Stiff network: Polycaprolactone (PCL) was chosen for the stiff network as it is a widely used biomaterial in scaffold fabrication, especially as a melt-extrusion polymer for accurate 3D-printing. It is also water insoluble and slow-degrading, ensuring that it will remain present throughout the repair process upon implantation. At an average molecular weight of 14,000, PCL has a melting point of 65° C.

Sacrificial network: Since the PCL and sacrificial material create interweaving networks and must therefore be printed concurrently layer-by-layer, the sacrificial network should ideally be comprised of a thermoplastic material with a melting point similar to that of PCL to minimize print time and temperature fluctations during melt extrusion. Yet the removal of the sacrificial material must also be relatively simple, non-toxic and rapid.

Bearing these considerations in mind, we've selected poly(ethylene glycol) (PEG) for the sacrificial material as it is a stiff polymer extensively used in cell culture applications and capable of being printed as a melt-extrusion polymer. At a molecular weight of 20,000, PEG has a melting point of 65° C., equal to that of the selected PCL material. Importantly, it is water soluble and can therefore be dissolved away through simple immersion in aqueous media.

Hydrogel material: Since the hydrogel must be cast within a micro-scale pore architecture and not printed, photocrosslinking is no longer an viable option: the hydrogel must be in a liquid, uncrosslinked form to be able to suffuse through the entire pore network. Only when complete suffusion occurs can this cell-carrying material be crosslinked into a hydrogel. Accordingly, ionic crosslinking was chosen to be the hydrogel crosslinking method. To retain the cytocompatible and cell-adhesive properties of gelatin as seen in previous examples, we've selected a mixture of gelatin and gellan gum (GG/gelatin) to be the basis for the hydrogel system. Indeed, while gelatin ensure cell-binding through its integrin motifs, gellan gum, another widely used biomaterial for cell encapsulation, ensures that crosslinking occurs in the presence of divalent cations, most notably Ca²⁺, which is found in culture medium solutions such as Minimum Essential Medium Eagle—Alpha Modification (αMEM). In addition, the combinatorial use of gelatin and gellan gum has previously been shown to generate stable composite hydrogels. Specifically, a composite formulation of 0.75% w/v gellan gum and 0.25% w/v gelatin generates a viscous liquid material at 37° C. which can be cast into the porous 3D-printed constructs and subsequently crosslinked in a 0.2 g/L calcium chloride solution such as αMEM.

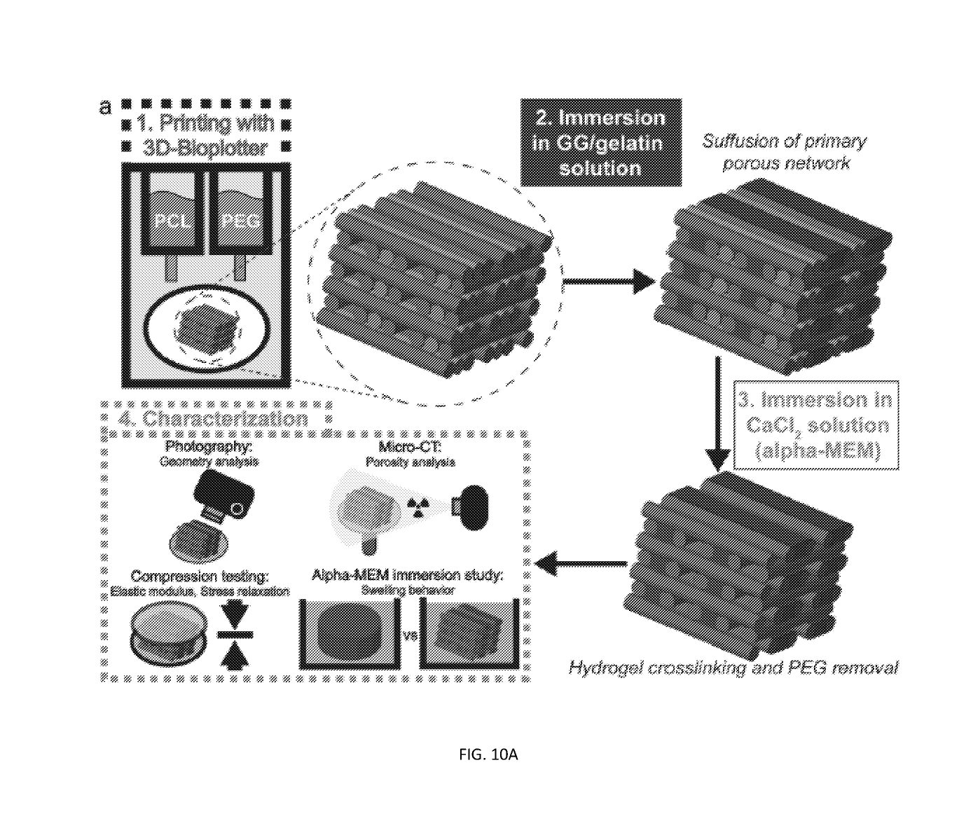

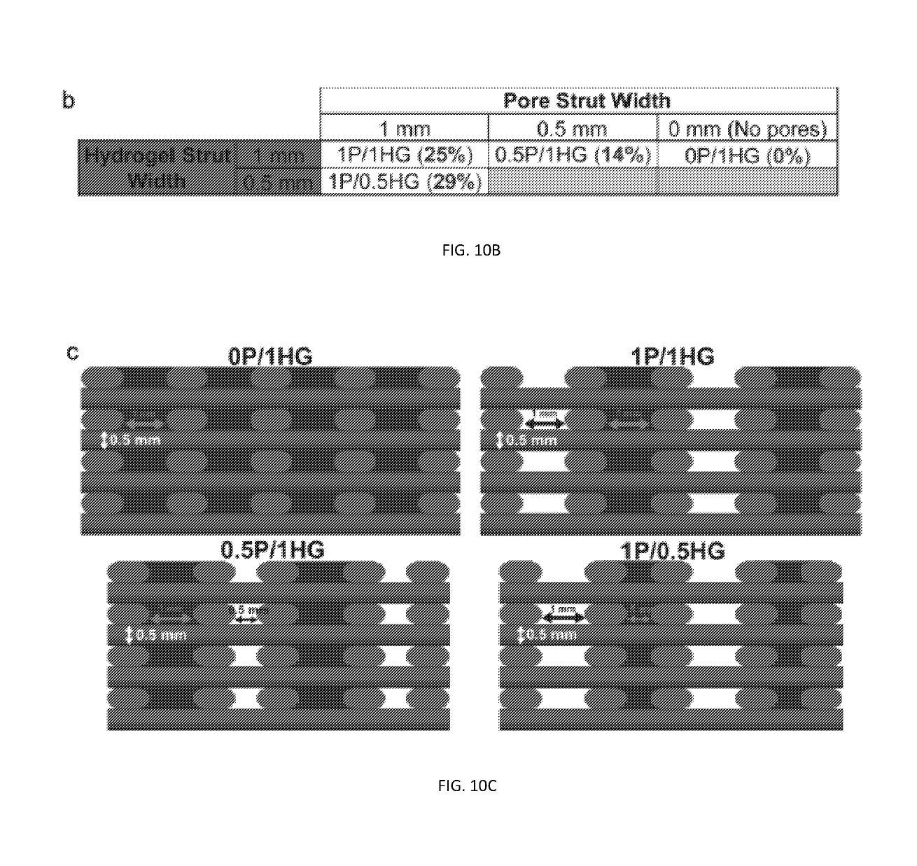

FIG. 10A(1-3) illustrates the biofabrication strategy developed in accordance with the previously described strategy and the selected materials. Experimental groups were generated by varying the widths of the pore struts (0 mm, 0.5 mm and 1 mm) as well as the widths of the hydrogel struts (0.5 mm and 1 mm), as shown in FIG. 10B-C. Generated constructs were subsequently characterized by photography to assess geometry, by micro-computed tomography (micro-CT) imaging to verify that each intended fabrication step is achieved, by compression testing to evaluate mechanical behavior, and by swelling testing to assess fluid flow behavior into the constructs (FIG. 10A(4)).

### Methods

i. 3D-Printing

Using an EnvisionTEC 3D-Bioplotter®, poly(ethylene glycol) (PEG; average Mn 20,000; Sigma) heated to 80° C. and polycaprolactone (PCL; average Mn 14,000; Sigma) heated to 90° C. were melt-extruded into porous hybrid constructs with a crosshatch architecture and a repeating PCL strut-pore channel-PCL strut-PEG strut pattern. The extrusion process was performed on matte paper and both PCL and PEG printing heads were fitted with stainless steel 24 G needles (300 μm inner diameter; Sigma). Printing speed was set to 3 mm/sec for the PCL head and 2 mm/sec for the PEG head. Extruded templates consisted of 10 layers, with each layer having a height of 0.5 mm. Both the widths of the primary pore channels and those of the PEG struts were varied to values of 0.5 mm and 1 mm by altering the dimensions of the computer generated 3D models imported into the 3D-Bioplotter software. The printed templates were subsequently sectioned into samples of size 5 mm×5 mm×5 mm using a surgical scalpel.

ii. Hydrogel Suffusion and PEG Removal

A composite (GG/gelatin) solution of 0.75% w/v gellan gum (GG) and 0.25% w/v gelatin was prepared by dissolving GELZAN™ CM and Type A, 300 bloom, porcine skin gelatin powders in deionized water at 37° C. under stirring. Sectioned samples were immersed in the prepared composite solution, which was subsequently allowed to cool to room temperature. After 15 minutes, samples were removed from the composite solution and immersed in Minimum Essential Medium Eagle—Alpha Modification (αMEM), which contains 0.2 g/L calcium chloride, for 2 hours at 37° C. in a stirring water bath to ensure PEG dissolution and crosslinking of the composite solution into a hydrogel.

iii. Construct Imaging and Width Analysis

Top and isometric photographs of samples from each experimental group both after printing and after sectioning were taken using a CANON POWERSHOTG11™ camera. The widths of the PCL struts, PEG struts and hydrogel channels were obtained using the scaling and measuring functions in ImageJ through manual endpoint selection over multiple struts and channels.

iv. Micro-Computed Tomography

Construct architecture was analyzed by micro-computed tomography using a calibrated desktop micro-CT scanner (SKYSCAN 1272™) at a voltage of 50 kV and a current of 200 μA. Four sectioned 1P/1HG constructs were scanned at an xyz resolution of 15 μm and an exposure time of 160 ms: one immediately after extrusion, a second after αMEM immersion over 2 hours, a third after immersion in a composite GG/G solution cooled to room temperature for physical gelation, and a fourth after immersion in a composite GG/G solution cooled to room temperature and subsequently in αMEM for 2 hours. Obtained isotropic slice data were reconstructed into 2D xy slice images, which were in turn compiled and analyzed to render 3D xyz images. Samples were reconstructed using a region of interest (ROI) with approximately 200 slices. Threshold levels were set to eliminate image noise and distinguish combined PCL, PEG and hydrogel material from pore regions. Porosities were determined using the software by selecting regions of interest which, in the xy plane, correspond to unit pattern elements of the constructs' repeating architecture.

v. Mechanical Testing

Final constructs from all experimental groups were placed on the compression platens of an INSTRON 4411™ Materials Testing Machine (INSTRON™ Ltd) in an unconfined setup, immersed in PBS, and preloaded with a compressive stress of 40 kPa prior to each test. The performed stress relaxation test consists of an initial uniaxial compression portion to a strain of 5% at a rate of 0.5% per second, followed by dwelling at that strain for 2 minutes (stress relaxation portion). Linear regression was performed on the obtained stress-strain data over the initial 1% of strain to obtain the slope of the initial linear portion of the stress-strain curve (Young's modulus). Exponential fitting of the stress relaxation portion of the data was performed using equation (4), where σ represents stress, aᵣₑₗₐₓ corresponds to the change in stress caused by relaxation while bᵣₑₗₐₓ corresponds to the equilibrium stress value reached over time. τ is a time constant that corresponds to the amount of time it takes for the stress to reach approximately 37% of its final value (1/e).

$$σ = a relax {e}^{- \frac{t}{τ}} + b relax \qquad ( 4 )$$

From the fitting, time-dependent mechanical behavior was quantified using the total change in stress during relaxation (−aᵣₑₗₐₓ), the total change in stress as a percentage of the initial stress prior to relaxation (aᵣₑₗₐₓ/(aᵣₑₗₐₓ+bᵣₑₗₐₓ)×100%) and the average stress rate, which corresponds to the average rate of change of stress over the initial 99% of stress relaxation.

vi. Swelling Test

A swelling kinetics study was performed to assess differences in hydrogel swelling in the presence of the stiff PCL network and in unconstrained conditions. Fully prepared 0P/1HG constructs were dried over a period of 1 week. Given the known initial weight concentration of the GG/gelatin hydrogel and assuming the measured decrease in weight during drying corresponds to the initial water weight of the GG/gelatin hydrogel material contained within the constructs, plain weight-matched GG/gelatin hydrogels were also prepared and dried over a period of 1 week. Both 0P/1HG constructs and plain hydrogel samples were subsequently immersed in 10 mL αMEM and weighed at multiple time points over 7 days of swelling. Swelling percentage was calculated using equation (5), where Mt corresponds to the hydrogel mass at time t and M₀ corresponds to the initial weight of the dried GG/gelatin polymer prior to immersion in PBS.

$$Swelling percentage = \frac{Mt - M 0}{M 0} \times 100 \% \qquad ( 5 )$$

vii. Statistics

One-way ANOVA with post-hoc Tukey analysis was performed to determine statistical significance between groups in the strut/channel width data and the mechanical testing data. A two-tailed t-test with Holm-Sidak correction for multiple comparisons was performed on the swelling percentage data. All graphs are shown as mean±SEM and the line width data is shown as mean±S.D., with n=8 for the strut/channel width data, n=1 for the porosity data from micro-CT imaging, n=6 for the mechanical testing data and n=7 for the swelling data. All graphs were plotted using GRAPHPAD PRISM 6™ software. A p-value of less than 0.05 was considered statistically significant.

### Results

i. Construct Geometry Assessment

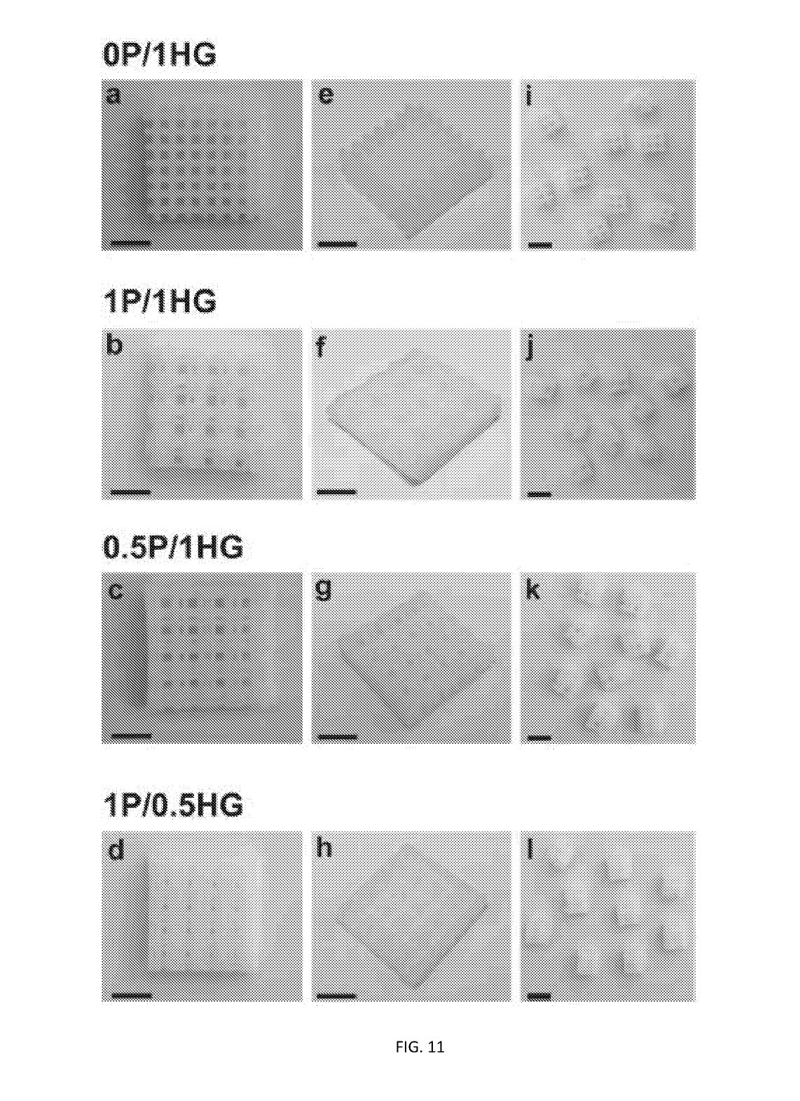

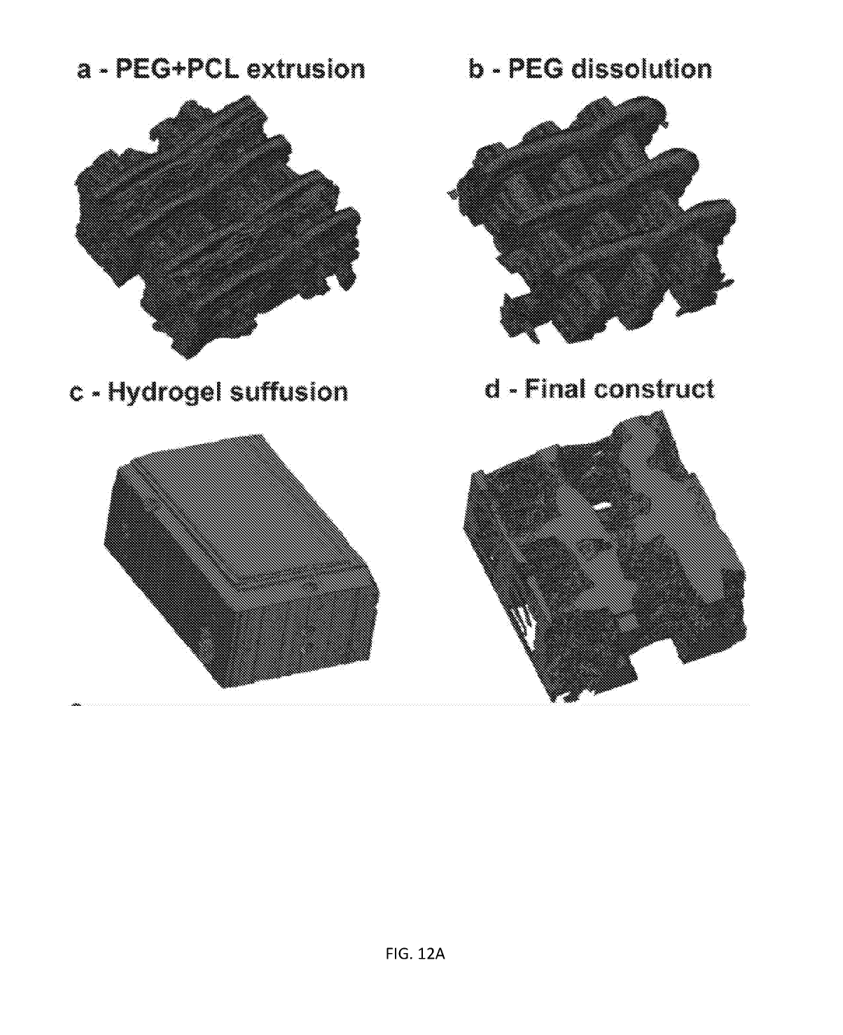

Immediately after the 3D-printing of porous hybrid constructs from all four experimental groups, geometric analysis was performed by photography, as seen in FIG. 11, to assess printing fidelity and consistency. Recorded measurements, shown in Table 4, include PCL strut widths, PEG strut widths and the widths of primary porous network channels to be filled with hydrogel. Overall, measurements remained close to intended values, with means not straying from target by more than 0.16 mm. Given the low variation in values for measurements from each group, which is indicative of high consistency, the observed differences between intended and targeted values are most likely the result of offsets in the width of elemental filaments between the computer models and actual extrusions.

TABLE 4
Widths of PCL struts, PEG struts and hydrogel channels
for all experimental groups calculated from obtained
images. Data shown as mean ± S.D.

| Group | PCL struts | PEG struts | Hydrogel channels |
|---|---|---|---|
| 0P/1HG | 0.928 (±0.0417) | ND | 1.064 (±0.0435) |
| 1P/1HG | 0.930 (±0.127) | 1.158 (±0.0989) | 1.093 (±0.0738) |
| 0.5P/1HG | 0.871 (±0.105) | 0.646 (±0.0581) | 1.034 (±0.143) |
| 1P/0.5HG | 0.857 (±0.101) | 1.024 (±0.0727) | 0.542 (±0.120) |

ii. Porosity Assessment by Micro-CT

To ensure that the construct preparation steps occurred as anticipated, a single 1P/1HG print was sectioned into four constructs, one of which remained unchanged while the other three were subjected to different steps of the preparation process, including (1) immersion in aqueous media (αMEM) to verify complete PEG dissolution, (2) immersion in a GG/gelatin solution and crosslinking in αMEM to ensure complete hydrogel suffusion, and (3) immersion in a GG/gelatin solution followed by immersion in αMEM to confirm final construct formation. Each construct was scanned using micro-CT and, since the described steps amount to material additions and removals with associated volumetric changes, the success of each preparation step was evaluated by comparing the porosity measurement from each construct against the corresponding expected value. Overall, measured porosity percentages did not stray by more than 11% from targeted values, indicating that both PEG dissolution and hydrogel suffusion were complete and successful when carried out both separately and sequentially, though more extensive studies are required to confirm this finding.

iii. Mechanical Properties

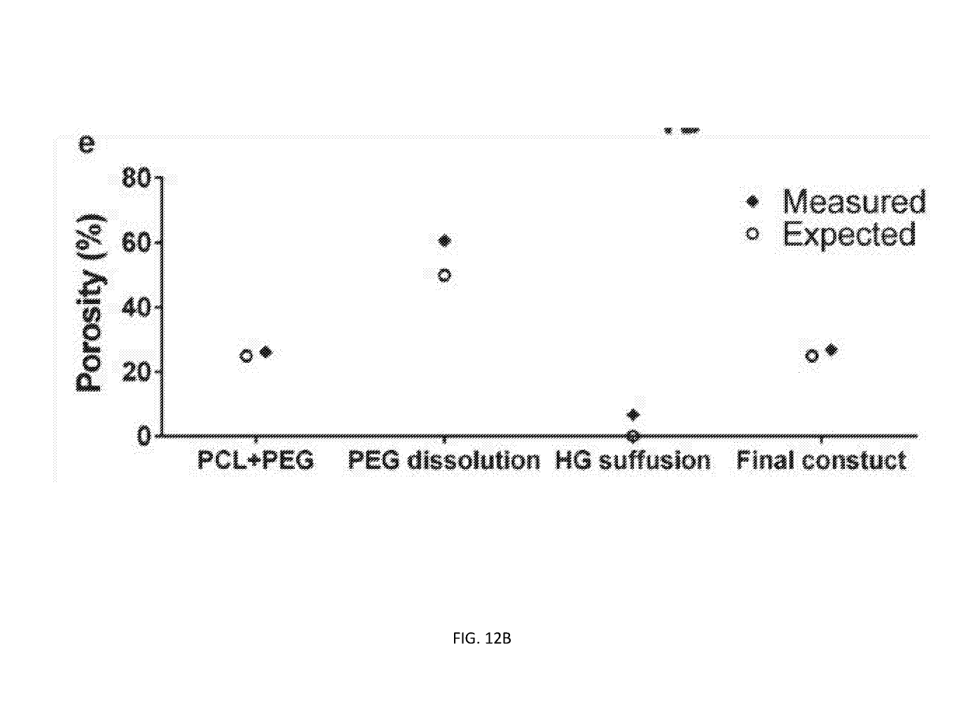

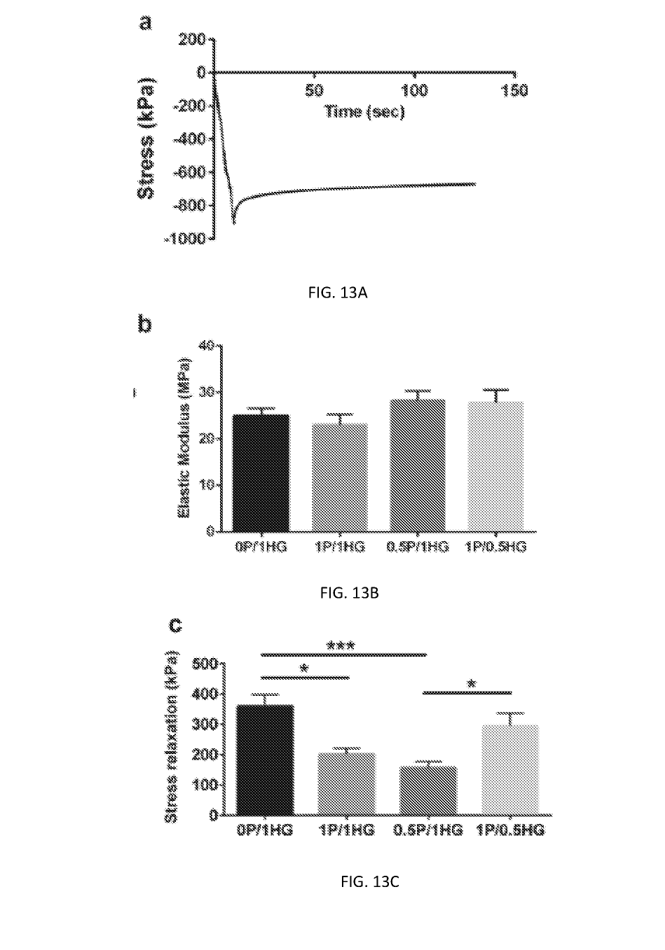

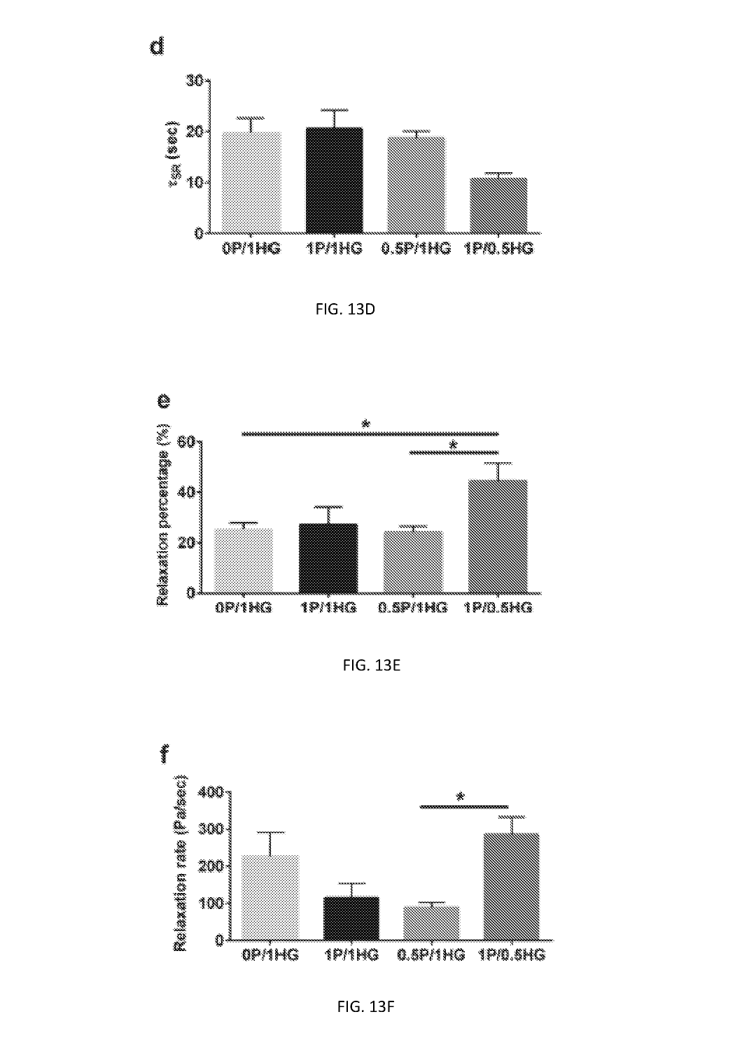

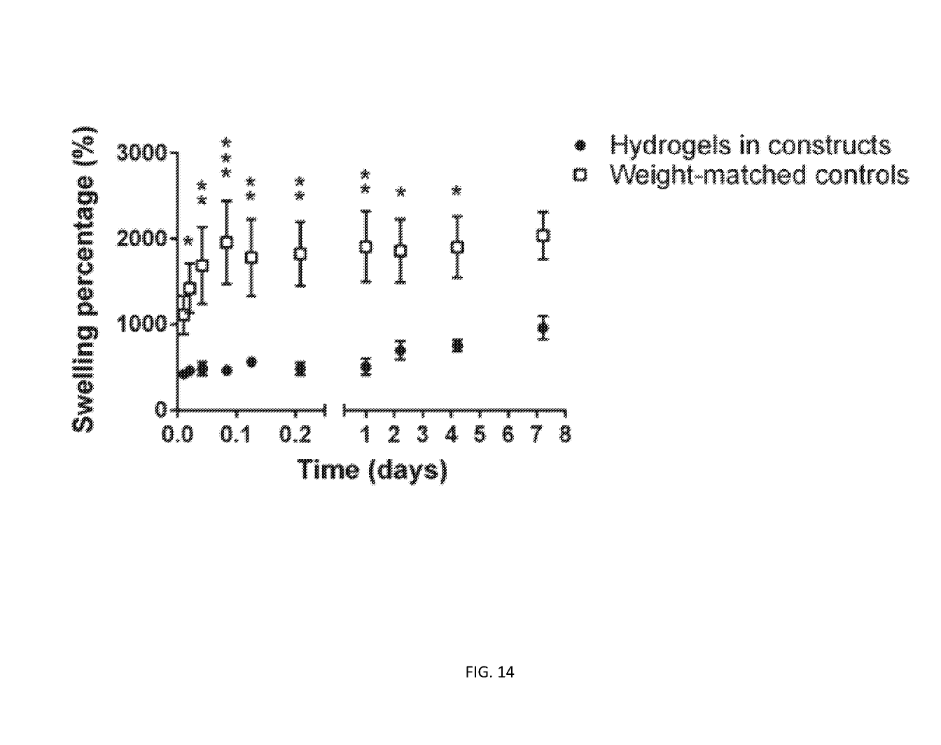

To evaluate both bulk elastic and time-dependent mechanical properties of the generated templates, final constructs from all four experimental groups were subjected to stress relaxation testing under unconfined hydrated testing, whereby strain was linearly increased to 5% and held constant for 2 minutes (FIG. 13A). No failure was observed in any of the constructs during and after testing. Elastic modulus measurements, calculated from the stress and strain data obtained during the linear increase in strain, were quite similar across all groups, as shown in FIG. 13B, with a global average of 26.3 (±1.14) MPa.

Time-dependent mechanical behavior was also quantified using exponential regressions of the relaxation portion of the stress vs time data. Specifically, the extent of stress change both alone (FIG. 13C) and as a percentage of stress immediately prior to relaxation (FIG. 13E), the time constant ti indicative of the time scale of relaxation (FIG. 13D), and the average rate of stress change over the initial 99% of relaxation (FIG. 13F) were calculated. Though no significant differences were found across groups for τ, values for the 1P/0.5HG group were markedly and consistently higher than values for the 0.5P/1HG group across the three remaining metrics. In addition, there is a trend of differences isolating the 0P/1HG group from other groups. Indeed, the 0P/1HG group had a greater absolute change in stress during relaxation compared to the 1P/1HG and 0.5P/1HG groups as well as a lower change in stress as a percentage of initial value during relaxation compared to the 1P/0.5HG group.

To probe the impact of the reinforcing stiff PCL network in the rate and extent of fluid flow into the GG/gelatin hydrogels, a swelling study was performed with dried 0P/1HG constructs and dried weight-matched hydrogel control samples (without a reinforcing stiff network) over the course of 7 days. Within a half hour of swelling, significant differences appear between the two groups and persist until day 4 with the control group exhibiting considerably greater swelling percentages, which confirms that hydrogel swelling is indeed constrained by a reinforcing stiff network. Interestingly, at day 7, though the mean swelling percentage of the control group was over two times greater than that of the 0P/1HG group, no significant difference was found between the two groups.

Characterization results for the generated constructs establish the proposed biofabrication strategy as a viable method of producing tailorable constructs for bone defect repair through endochondral ossification. Indeed, the developed fabrication strategy is capable of generating templates with great spatial resolution as well as tunable architectural and mechanical properties whilst still minimizing unwanted swelling deformation. In addition, the decision to cast the hydrogel material instead of extruding it will very likely be of benefit to encapsulated cells as they will not be subjected to the damaging shear stresses experienced during extrusion. Nevertheless, more extensive studies remain to be made to confirm findings and optimize the platform. For instance, though the printing process was shown to be fairly consistent as evidenced by the minimal variation in geometric measurements across replicates, some improvements could be made with printing fidelity by further harmonizing the widths of individual extrusion filaments with those of corresponding computer generated models. Moreover, additional replicates across all experimental groups are certainly required to confirm the obtained micro-CT results according to which each step of the fabrication process was successful.

Delving into the mechanical properties of the constructs, it is reasonable to assume that, discounting volumetric composition, since the compressive modulus of polycaprolactone, which is recorded to be around 40 MPa, is markedly higher than that of the hydrogel material, which would be in the order of 0.1 MPa as supported by findings from previous examples, polycaprolactone would be the primary determinant of elastic modulus in these constructs. It therefore stands to reason that the experimental groups with the highest PCL content, namely 0.5P/1HG and 1P/0.5HG, would exhibit higher moduli compared to groups with lower PCL content, namely 0P/1HG and 1P/1HG. Yet surprisingly, though the means of the high PCL content groups were to be sure higher than those of the low PCL content groups, there was no significant difference between any of the groups. Although the presence of hydrogel material in the constructs might account for this, a more likely explanation can be found in the dimensions of the tested constructs, which were 5 mm×5 mm×5 mm. It is possible that, at this size, the PCL networks might have buckled under compression in such a way that the differences in PCL content between groups were not found to have had a significant impact on elastic modulus. This is supported by findings that properties such as elastic modulus are dependent on size for both PCL and other materials.

Contrary to the uniformity observed across experimental groups vis-à-vis elastic behavior, time-dependent mechanical behavior exhibited significant variability across groups for multiple metrics. The most crucial indicator in the elucidation of the primary mechanism behind stress relaxation in these constructs is the finding that differences were most consistently seen between the 0.5P/1HG and 1P/0.5HG groups. From a structural standpoint, though both groups have the same PCL content (57% by volume), they are the two most dissimilar groups in terms of the ratio of porosity to hydrogel content. Indeed, while the 0.5P/1HG group has a porosity of 14% and a volumetric hydrogel content of 29%, leading to a porosity-to-hydrogel content ratio of 0.5, the 1P/0.5HG group has a porosity of 29% and a volumetric hydrogel content of 14%, leading to a porosity-to-hydrogel content ratio of 2. For comparison, the porosity-to-hydrogel content ratio of the 1P/1HG group is 1. And since both the extent and rate of stress relaxation is greater in the 1P/0.5HG group compared to the 0.5P/1HG group, the likely mechanism of stress relaxation in the constructs during compression is the squeezing of hydrogel material into pore spaces which alleviates internal stresses. Since there is the most amount of pore space with respect to hydrogel material in the 1P/0.5HG group, it is therefore quite tenable that hydrogel material was displaced faster and to a greater extent into the pore network, leading to greater stress relaxation and an increased relaxation rate. Conversely, since there is the least amount of pore space with respect to hydrogel material in the 0.5P/1HG group, hydrogel material was squeezed slower and to a lesser extent into the pore network, leading to lower stress relaxation and a decreased relaxation rate. Another group with marked differences in time-dependent mechanical behaviour compared to others is the 0P/1HG group. This observation, compounded with the fact that there is no porosity in the 0P/1HG group, suggests that another mechanism is at play during relaxation under compression in this group. Looking closer, the finding that the 0P/1HG group had the greatest absolute change in stress yet the second lowest change in stress as a percentage of initial value indicates that great stresses were accumulated prior to relaxation during the linear increase in strain. This is most probably because there were no stress-alleviating pores into which hydrogel material could have been forced into. The ensuing hypothesis is therefore that stress relaxation was achieved in the 0P/1HG constructs through the simple displacement of hydrogel material outside of the construct boundaries. The proposed conjecture for stress alleviation through squeeze deformation has been explored in previous studies, examples of which include mechanical characterizations of hydrogels for cartilage and nucleus pulposus engineering, lending additional credence to this hypothesis.

Finally, the swelling study results serve to confirm another method by which swelling deformation may be constrained using the developed biofabrication strategy: the presence of a reinforcing stiff network reduces the rate and extent of fluid flow into hydrogels by acting as a physical barrier to increasing hydrogel volume. Interestingly, as previously observed, no significant difference was found at day 7 between the swelling percentages of the 0P/1HG and control groups and the mean swelling percentage of the control group was over two times greater than that of the 0P/1HG group. A possible explanation for this is that between days 4 and 7, the hydrogel material fully enveloped the stiff network in the 0P/1HG constructs, thereby eliminating the ensuing effectiveness of the stiff network at constraining swelling.

Overall, a follow-up comprehensive investigation is required to validate the interesting findings and conjectures regarding the fabricated constructs, especially with respect to mechanical and swelling behavior, given the limitations of the conducted study. Firstly, additional tests are required to probe the veracity of the advanced hypotheses, including testing with constructs of greater size, creep testing to confirm stress relaxation findings, and visualization of hydrogel squeeze/swell-based deformation through optical microscopy or 3D imaging methods. The study could also benefit from complementing mathematical models (ex: a finite element analysis of the stress relaxation tests) which could account for key findings. Finally, cell studies are needed to investigate whether cell encapsulation alters any construct properties. Once such an extensive study is completed, viability, tissue differentiation and pre-clinical animal implantation studies are warranted to fully confirm product feasibility and to investigate how parameter modulation impacts the efficiency of the constructs with respect to bone defect repair. Given that the porosity-to-hydrogel content ratio is lowest for the 0.5P/1HG group and that hypoxia has been widely shown to promote chondrogenesis, it is likely a priori that the 0.5P/1HG group will generate tissue most similar to cartilage and will therefore perform better upon implantation.

### Evaluation of Proposed Strategy and Conclusions

i. Criteria/Constraint Satisfaction

At the conclusion of the study, success or failure of the developed biofabrication strategy was gauged with respect to each previously established criteria and constraint:

Bulk size and shape for targeted defect types: Though construct characterization was performed at the spatial scale of unit elements of the PCL, hydrogel and pore network architectures, the use of additive manufacturing and the structural integrity that the stiff network provides ensure that the generated constructs may be scaled up and shaped to conform to any non-union defect along the mid-section of long bones.

Surgical fixation: It was found that during mechanical testing, none of the constructs fractured at strains of up to 5%, which exceeds the 1% limit criteria previously set and confirms that the generated templates will not fail during implantation upon press fitting and plate fixation.

Elastic modulus: With a global mean of 26.3 (±1.14) MPa and fairly uniform measurements across replicates and groups, the generated constructs are acceptably within the 5-30 MPa range established to prevent stress shielding and ensure mechanotransduction while adequately supporting press fitting.

Printing precision and consistency: The greatest deviation in width mean from target value was found to be 29% (9% greater than the set criteria value) while the largest relative standard deviation for any given group of measurements was reported to be 22% (2% greater than the set criteria value). The employed additive manufacturing platform therefore narrowly misses both the targeted precision and consistency levels required to produce either distinct or identical multi-material constructs as needed. Thus, as previously discussed, printing fidelity should be further improved by harmonizing the widths of individual extrusion filaments with those of corresponding computer generated models. In addition, minor improvements stand to be made in terms of ensuring geometric uniformity across multiple iterations of the same printing task. This can be accomplished by further standardizing environmental conditions, which include ambient temperature, air convection and the amount of loaded material within extrusion cartridges, across all prints.

Construct parameter modulation: By successfully modulating pore and hydrogel strut widths in the construct architecture, the developed fabrication strategy was able to generate constructs with a variety of porosities exceeding the set minimal range of 20% and with marked differences in time-dependent mechanical behavior, all of which confirms the tailorability of generated constructs through parameter modulation.

Stiff material content: The maximal volumetric PCL content in the fabricated scaffolds, which was 57%, is also acceptably under the established threshold of 70%, which lends support to the prediction that the generated templates will support adequate vascularization.

Swelling: In addition to the reduction in swelling achieved through the successful elimination of hydrogel extrusion from the fabrication process, the incorporation of a stiff network also participated in further constraining swelling. Indeed, until day 5 of swelling, the highest recorded swelling percentage for hydrogels within the stiff network was 954%, which is less than the set maximal limit of 962%. These findings lend credence to the expectation that deformation due to swelling will be minimized.

Thus, overall, the developed fabrication strategy met all of the previously established criteria and constraints.

Any document listed herein is hereby incorporated herein by reference in its entirety. While these developments have been disclosed with reference to specific embodiments, it is apparent that other embodiments and variations of this invention are devised by others skilled in the art without departing from the true spirit and scope of the developments. The appended claims include such embodiments and variations thereof.

1. An engineered porous cartilage template having a bone-mimicking internal structure.

2.-18. (canceled)

19. A composition comprising the porous cartilage template according to claim 1 and mesenchymal stem cells (MSCs).

20. (canceled)

21. (canceled)

22. A composition comprising the porous cartilage template according to claim 1 and chondrocytes.

23.-26. (canceled)

27. A method of promoting the repair of a bone defect in a patient, the method comprising:
preparing a porous cartilage template having a bone-mimicking internal structure,
embedding a plurality of cells into the porous cartilage template, and
implanting the porous cartilage template into the bone defect in the patient, thereby promoting the repair of the bone defect.

28. The method of claim 27, further comprising a step of stabilizing the bone defect.

29. The method according to claim 28, wherein the step of stabilizing the bone defect comprises emergency surgery to immobilize the bone defect by the insertion of one or more selected from the group consisting of: compression plates, rods, nails, Kirschner wires, and casts.

30. The method according to claim 27 wherein the porous cartilage template is prepared by 3D-printing.

31. The method of claim 30, wherein the 3D-printing is based on imaging data acquired from a bone defect in the patient.

32. The method according to claim 31, wherein the imaging data is acquired by computed tomography (CT) scan or magnetic resonance imaging.

33. The method according to claim 27, wherein the plurality of cells comprises mesenchymal stem cells.

34. The method according to claim 33, wherein the mesenchymal stem cells are harvested from the patient.

35. The method according to claim 27 wherein the plurality of cells comprises chondrocytes.

36. The method according to claim 27, wherein the 3D-printing and embedding steps are performed simultaneously.

37. The method according to claim 36, wherein the plurality of cells is contained in a hydrogel that is 3D-printed to form at least a portion of the porous cartilage template.

38. The method according to claim 27, further comprising culturing the plurality of cells to produce mature cartilage.

39. The method according to claim 38, wherein the plurality of cells are mesenchymal stem cells and further comprising differentiating the mesenchymal stem cells into chondrocytes.

40. The method according to claim 27, wherein the porous cartilage template is secured in the bone defect by press fitting.

41. A method of preparing a porous cartilage template for bone repair, the method comprising:
3D-printing a porous network based on bone imaging data, the porous network comprising:
a support component;
a sacrificial component; and
a plurality of pores;
casting a cell-carrier component comprising a plurality of cells into the plurality of pores,
evacuating the sacrificial component to form a network of passages among the support component and cell-carrier component; and
culturing the plurality of cells of cells to form mature cartilage;
thereby forming the porous cartilage template.

42.-50. (canceled)

51. The method according to claim 41, further comprising a step of crosslinking the cell-carrier component.

52. (canceled)

53. (canceled)

54. The method according to claim 41, wherein the sacrificial component is evacuated by dissolution in aqueous solution.
